SEQUENCE LISTING (26 sequences)

SEQ ID NO: 1 - DNA, 31 bp, Artificial Sequence
  primer, oligo-LAG3-F-EcoRI
ggaattcatgtgggaggctcagttcctgggc

SEQ ID NO: 2 - DNA, 28 bp, Artificial Sequence
  primer, oligo-LAG3-R-5P
agtgaggttatacatgatggagacgttg

SEQ ID NO: 3 - DNA, 21 bp, Artificial Sequence
  primer, oligo-LAG3-F-5P
ctccagccaggggctgaggtc

SEQ ID NO: 4 - DNA, 30 bp, Artificial Sequence
  primer, oligo-LAG3-R-SpeI
gactagttgggggctccagacccagaacag

SEQ ID NO: 5 - DNA, 28 bp, Artificial Sequence
  primer, hIgG-F-SpeI
gagtagtgcagagcccaaatcttgtgac

SEQ ID NO: 6 - DNA, 34 bp, Artificial Sequence
  primer, hIgG-R-XbaI
gctctagagctcatttacccggagacagggagag

SEQ ID NO: 7 - DNA, 1503 bp, Homo sapiens
  sig_peptide (1)..(66)
  CDS (1)..(1500): LAG3/Fc
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactagtgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 8 - protein, 500 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 2211 bp, Homo sapiens
  sig_peptide (1)..(66)
  CDS (1)..(2208): LAG3-LAG3/Fc
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactagtgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 10 - protein, 736 aa, Homo sapiens [sequence omitted]

SEQ ID NO: 11 - DNA, 1473 bp, Homo sapiens
  CDS (1)..(1470): TNFR2/Fc
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctcctccttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 490 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSSFLYSKLTVDKSRWQQGNVFSCSVMHEALHNHYTQKSLSLSPGK

SEQ ID NO: 13 - DNA, 2163 bp, Homo sapiens
  CDS (1)..(2160): TNFR2-TNFR2/Fc
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcggatccaacgcaactacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctcctccttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 720 aa, Homo sapiens [sequence omitted]

SEQ ID NO: 15 - DNA, 1314 bp, Homo sapiens
  CDS (1)..(1311): CD2/Fc
atgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtcctgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 16 - protein, 437 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1854 bp, Homo sapiens
  CDS (1)..(1851): CD2-CD2/Fc
atgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtcctaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtcctgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtctgtcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 617 aa, Homo sapiens [sequence omitted]

SEQ ID NO: 19 - DNA, 1134 bp, Homo sapiens
  CDS (1)..(1131): CTLA4/Fc
atgaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 377 aa, Homo sapiens
MRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1509 bp, Homo sapiens
  CDS (1)..(1506): CTLA4-CTLA4/Fc
atgaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattcggataacatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtctgtcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 502 aa, Homo sapiens
MRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDNMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1335 bp, Homo sapiens
  CDS (1)..(1332): TNFR1/Fc
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctcctccttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 24 - protein, 444 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSSFLYSKLTVDKSRWQQGNVFSCSVMHEALHNHYTQKSLSLSPGK

SEQ ID NO: 25 - DNA, 2028 bp, Homo sapiens
  CDS (1)..(2025): TNFR2-TNFR1/Fc
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcggatccgggaacatttcactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctcctccttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 675 aa, Homo sapiens [sequence omitted]

(19) United States
(12) Patent Application Publication
(10) Pub. No.: US 2007/0110746 A1
(43) Pub. Date: May 17, 2007

(54) Pharmaceutical composition for treatment of immunological disorders

(76) Inventor: Yong-Hoon Chung, Seoul (KR)

Correspondence Address: SHERIDAN ROSS PC, 1560 BROADWAY, SUITE 1200, DENVER, CO, 80202, US

(21) Appl. No.: 10/539,946

(22) PCT Filed: Feb. 18, 2005
(86) PCT No.: PCT/KR05/00457

§ 371 Date: Dec. 26, 2006

(51) Int. Cl. A61K 39/395; C07K 16/28

(52) U.S. Cl. 424/144.1; 530/388.22

(57) ABSTRACT

Disclosed is a pharmaceutical composition for treating immunological disorders by inhibiting the activation of T lymphocytes, comprising, as active ingredients, two or more selected from the group consisting of a substance capable of blocking binding of an MHC (Major Histocompatibility Complex) Class II molecule and a receptor thereof, a substance capable of blocking binding of a costimulatory molecule and a receptor thereof, a substance capable of blocking binding of an adhesion molecule and a receptor thereof, and a substance capable of blocking binding of a cytokine and a receptor thereof.

### TECHNICAL FIELD

The present invention relates to a pharmaceutical composition for treating immunological disorders by inhibiting the activation of T lymphocytes, comprising, as active ingredients, two or more selected from the group consisting of: a substance capable of blocking binding of an MHC (Major Histocompatibility Complex) Class II molecule and a receptor thereof; a substance capable of blocking binding of a costimulatory molecule and a receptor thereof, a substance capable of blocking binding of an adhesion molecule and a receptor thereof, and a substance capable of blocking binding of a cytokine and a receptor thereof.

### BACKGROUND ART

Immune responses are processes that protect the self from the non-self, such as various impurities, bacteria or viruses. The immune system is elaborately designed not to attack the self. However, in some cases, these immune responses attack the self and damage the body, representative examples of which are the immunological rejection of transplanted organs or tissues and autoimmune diseases.

In treatment of diseases caused by organ or tissue transplantation, the most significant problem concerns severe transplantation rejection in recipients, which occurs after the transplantation of tissues or organs from donors. Transplantation rejection refers to immune responses in a recipient which try to eliminate a graft from a donor whose genetic background is different from that of the recipient because the recipient recognizes the graft as a foreign substance. This transplant rejection occurs due to a complicated cooperation of cellular immunity mediated by T lymphocytes and humoral immunity mediated by antibodies, but is mainly due to cellular immunity mediated by T lymphocytes.

One method for treating transplantation rejection involves employing chemical compounds suppressing the activity of T lymphocytes. Such immunosuppressive agents include mizoribine (MZ), cyclosporin (CsA), tacrolimus (FK-506), azathioprine (AZ), leflunomide (LEF), adrenocortical steroids such as predonisolone or methylpredonisolone, deoxypergualin (DGS), and sirolimus.

PCT Publication No. WO 1999/65908 discloses a method of treating autoimmune diseases using pyrrolo [2,3] pyrimidine compounds as immunosuppressive agents. PCT Publication No. WO 2000/21979 discloses a method of treating transplant reaction or autoimmune diseases using cyclic tetrapeptide compounds. On the other hand, in some cases, immune cells do not distinguish between the self and the non-self (foreign) materials and attack the self, and this phenomenon is called “autoimmunity”. Autoimmune responses may cause disorders in all areas of the body. Examples of autoimmune diseases include rheumatoid arthritis, multiple sclerosis, myasthenia gravis, Grave's disease, Hashimoto's thyroiditis, Addison's disease, vitilligo, scleroderma, Goodpasture syndrome, Becet's disease, Crohn's disease, ankylosing spondylitis, uveitis, thrombocytopenic purpura, pemphigus vulgaris, childhood diabetes, autoimmune anemia, cryoglobulinemia, adrenoleukodystrophy (ALD), and systemic lupus erythematosus (SLE).

PCT Publication No. WO 1996/40246 describes a method of treating and preventing T cell-mediated autoimmune diseases, such as multiple sclerosis. The method comprises administering to a subject a therapeutically or prophylactically effective amount of an antagonist of a receptor on the surface of T cells, which mediate contact-dependent helper effector functions. The antagonist is an antibody or a fragment thereof which specifically binds to the T cell receptor gp39.

PCT Publication No. WO 2002/22212 discloses a method of treating autoimmune diseases, preferably B cell-mediated autoimmune diseases, using the combination of at least one immunoregulatory antibody and at least one B cell depleting antibody, for example, an antibody that targets CD19, CD20, CD22, CD23 or CD37.

However, the aforementioned compounds cause significant adverse effects when used for treating immunological disorders, so that they have limited applications. As described in PCT Publication No. WO 1996/40246, when an antibody is administered alone, desired therapeutic efficacy is difficult to achieve. Also, since autoimmune diseases or transplantation rejection begin with activation of T lymphocytes, the blocking of B cell functions as described in PCT Publication No. WO 2002/22212 does not lead to effective inhibition of immune responses.

### DISCLOSURE OF THE INVENTION

Leading to the present invention, intensive and thorough research into the development of more effective immunosuppressive agents, conducted by the present inventors, resulted in the finding that, when proteins selected from at least two of several groups of proteins that participate in activating T lymphocytes are simultaneously blocked, the activity of T lymphocytes is effectively suppressed in comparison with known methods.

In one aspect, the present invention provides a pharmaceutical composition for treating immunological disorders by inhibiting the activation of T lymphocytes, comprising, as active ingredients, two or more selected from the group consisting of: a substance capable of blocking binding of an MHC Class II molecule and a receptor thereof, a substance capable of blocking binding of a costimulatory molecule and a receptor thereof, a substance capable of blocking binding of an adhesion molecule and a receptor thereof, and a substance capable of blocking binding of a cytokine and a receptor thereof.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a genetic map of a recombinant expression plasmid pCD22Ig expressing a concatameric fusion monomeric protein CD2-CD2/Fc according to the present invention;

FIG. 2 is a genetic map of a recombinant expression plasmid pCT44Ig expressing a concatameric fusion monomeric protein CTLA4-CTLA4/Fc according to the present invention;

FIG. 3 is a genetic map of a recombinant expression plasmid pLAG33Ig expressing a concatameric fusion monomeric protein LAG3-LAG3/Fc according to the present invention;

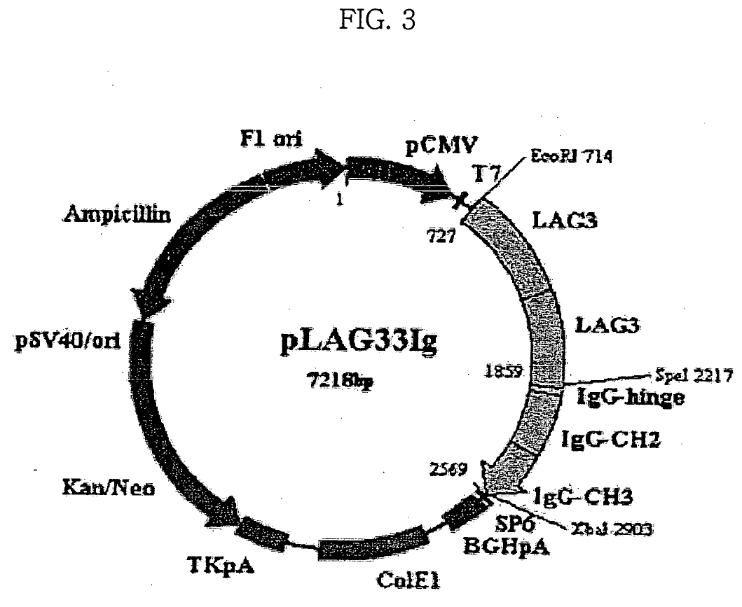

FIG. 4 is a genetic map of a recombinant expression plasmid pTR21Ig-Top' xpressing a concatameric fusion monomeric protein TNFR2-TNFR1/Fc according to the present invention;

FIG. 5a shows the results of SDS-PAGE analysis of simple fusion dimeric proteins ([CD2/Fc]₂, [CTLA4/Fc]₂ and [LAG3/Fc]₂) and concatameric fusion dimeric proteins ([CD2-CD2/Fc]_{2,} [CTLA4-CTLA4/Fc]₂ and [LAG3-LAG3/Fc]₂) according to the present invention;

FIG. 5b shows the results of SDS-PAGE analysis of simple fusion dimeric proteins (1:[TNFR1/Fc]₂, 2: [TNFR2/Fc]₂) and concatameric fusion dimeric proteins (3:[TNFR2-TNFR1]/Fc]₂, 4: [TNFR2-TNFR2]/Fc)₂) according to the present invention;

FIG. 6a is a graph showing the inhibitory effects of simple fusion dimeric proteins ([TNFR2/Fc]₂, [CD2/Fc]₂, [CTLA4/Fc]₂ and [LAG3/Fc]₂) according to the present invention on T lymphocyte proliferation;

FIG. 6b is a graph showing the inhibitory effects of combinations of simple fusion dimeric proteins according to the present invention, [CTLA4/Fc]₂+[TNFR2/Fc]₂, [CTLA4/Fc]₂+[CD2/Fc]₂ and [CTLA4/Fc]₂+[LAG3/Fc]₂ as well as [CTLA4/Fc]₂ alone on T lymphocyte proliferation;

FIG. 6c is a graph showing the inhibitory effects of concatameric fusion dimeric proteins ([TNFR2-TNFR2/Fc]₂, [CD2-CD2/Fc]₂, [CTLA4-CTLA4/Fc]₂ and [LAG3-LAG3/Fc]₂), according to the present invention, on T lymphocyte proliferation;

FIG. 6d is a graph showing the inhibitory effects of combinations of concatameric fusion dimeric proteins according to the present invention, [CTLA4-CTLA4/Fc]₂+[TNFR2-TNFR2/Fc]₂, [CTLA4-CTLA4/Fc]₂+[CD2-CD2/Fc]₂ and [CTLA4-CTLA4/Fc]₂+[LAG3-LAG3/Fc]₂, as well as [CTLA4-CTLA4/Fc]₂ alone on T lymphocyte proliferation;

FIG. 7a is a graph showing the reducing effects of simple fusion dimeric proteins ([TNFR2/Fc]₂, [CD2/Fc]₂, [CTLA4/Fc]₂ and [LAG3/Fc]₂) according to the present invention on the severity of collagen-induced arthritis (CIA) in mice;

FIG. 7b is a graph showing the reducing effects of combinations of simple fusion dimeric proteins according to the present invention, [CTLA4/Fc]₂+[TNFR2/Fc]₂, [CTLA4/Fc]₂+[CD2/Fc]₂ and [CTLA4/Fc]₂+[CD2-CD2/Fc]₂ as well as [CTLA4/Fc]₂ alone on the severity of CIA in mice;

FIG. 7c is a graph showing the reducing effect of concatameric fusion dimeric proteins ([TNFR2-TNFR2/Fc]₂, [CD2-CD2/Fc]₂, [CTLA4-CTLA4/Fc]₂ and [LAG3-LAB3/Fc]₂) according to the present invention on the severity of CIA in mice;

FIG. 7d is a graph showing the reducing effects of combinations of concatameric fusion dimeric proteins according to the present invention, [CTLA4-CTLA4/Fc]₂+[TNFR2-TNFR2/Fc]₂, [CTLA4-CTLA4/Fc]₂+[CD2-CD2/Fc]₂ and [CTLA4-CTLA4/Fc]₂+[LAG3-LAG3/Fc]₂, as well as [CTLA4-CTLA4/Fc]₂ alone on the severity of CIA in mice;

FIG. 8a is a graph showing the improving effect of simple fusion dimeric proteins ([CD2/Fc]₂, [CTLA4/Fc]₂ and [LAG3/Fc]₂) according to the present invention on survival from graft-versus-host disease (GVHD) in mice;

FIG. 8b is a graph showing the improving effects of combinations of simple fusion dimeric proteins according to the present invention, [GTLA4/Fc]₂+[LAG3/Fc]₂ and [CD2/Fc]₂+[CTLA4/c]₂, on survival of graft-versus-host disease (GVHD) in mice;

FIG. 8c is a graph showing the improving effects of a simple fusion dimeric protein [CTLA4/Fc]₂ and a concatameric fusion dimeric protein [CTLA4-CTLA4/Fc]₂ according to the present invention on survival of graft-versus-host disease (GVHD) in mice;

FIG. 8d is a graph showing the improving effects of a simple fusion dimeric protein [TNFR2/Fc]₂ and a concatameric fusion dimeric protein [TNFR2-TNFR2/Fc]₂ according to the present invention on survival of graft-versus-host disease (GVHD) in mice;

FIG. 8e is a graph showing the improving effects of a simple fusion dimeric protein [TNFR2/Fc]₂ and concatameric fusion dimeric proteins, [TNFR2-TNFR1/Fc]₂ and [TNFR2-TNFR2/Fc]₂ according to the present invention on survival of graft-versus-host disease (GVHD) in mice; and

FIG. 8f is a graph showing the improving effects of concatameric fusion dimeric proteins, [CD2-CD2/Fc]₂, [CTLA4-CTLA4/Fc]₂ and [LAG3-LAG3/Fc]₂), and combinations thereof, [CD2-CD2/Fc]₂+[CTLA4-CTLA4/Fc]₂, on survival of graft-versus-host disease (GVHD) in mice.

### BEST MODE FOR CARRYING OUT THE INVENTION

The present invention relates to a pharmaceutical composition for treating immunological disorders by inhibiting the activation of T lymphocytes, comprising, as active ingredients, two or more selected from the group consisting of: a substance capable of blocking binding of an MHC Class II molecule and a receptor thereof, a substance capable of blocking binding of a costimulatory molecule and a receptor thereof, a substance capable of blocking binding of an adhesion molecule and a receptor thereof, and a substance capable of blocking binding of a cytokine and a receptor thereof.

As known in the art, T lymphocytes recognize only antigens that associate with “MHC (Major Histocompatibility Complex) Class II molecules” on the surface of antigen presenting cells, and are subsequently activated and cause immune responses against the antigens. In addition to MHC Class II molecules, other molecules delivering activation signals to T lymphocytes are present on antigen presenting cells, and these molecules are called “costimulatory molecules”. Also, so-called “adhesion molecules” function to strengthen intercellular adhesiveness between antigen presenting cells and T lymphocytes with the function to deliver signals. Further, various “cytokines” participate in immune responses including T cell activation.

The “MHC Class II molecules” initiate the activation of T lymphocytes, and their receptors include CD4 and LAG3. MHC Class II molecules bind to antigens and then are recognized by their receptor (CD4) on the surface of T lymphocytes, leading to the activation of T lymphocytes. Thus, this function of MHC Class II molecules may be suppressed by blocking the binding between MHC Class II molecules and their receptors. Substances capable of displaying such suppressive action include, but are not limited to, antibodies to MHC Class II molecules and receptors of MHC Class II molecules in free forms. Herein, the free MHC Class II receptors include all receptors that are capable of specifically binding to MHC Class II molecules, and preferably are Ig fusion proteins in which MHC Class II receptors or soluble extracellular domains thereof are linked to whole immunoglobulins or Fc fragments thereof. Further, the Ig fusion proteins may be in additionally glycosylated forms.

The “costimulatory molecules” include B7 (B7.1 and B7.2), CD154, CD70, 0X40L, ICOS-L, 4-1BBL, HVEM, FASL and PDL (PDL-1 and PDL-2), and their receptors include CD28 and CTLA4, CD40, CD27, 0X40, ICOS, 4-1BB (CD137), LIGHT, FAS (CD95) and PD-1, respectively. Costimulatory molecules are expressed on the surface of antigen presenting cells, and bind to their receptors expressed on the surface of T lymphocytes, leading to the activation of T lymphocytes. Thus, T cell activation by costimulatory molecules may be suppressed by blocking the binding between costimulatory molecules and their receptors. Substances capable of displaying such suppressive action include, but are not limited to, antibodies to costimulatory molecules and receptors of costimulatory molecules in free forms. Herein, the free receptors of costimulatory molecules include all receptors that are capable of specifically binding to costimulatory molecules, and preferably are Ig fusion proteins in which receptors of costimulatory molecules or soluble extracellular domains thereof are linked to immunoglobulins or Fc fragments thereof. Further, the Ig fusion proteins may be in additionally glycosylated forms.

The “adhesion molecules” include LFA-3, ICAM-1 and VCAM-1, and their receptors include CD2, LFA-1 and VLA-4, respectively. Adhesion molecules are expressed on the surface of antigen presenting cells, and bind to their receptors expressed on the surface of T lymphocytes, leading to the activation of T lymphocytes. Thus, T cell activation by adhesion molecules may be suppressed by blocking the binding between adhesion molecules and their receptors. Substances capable of displaying such suppressive action include, but are not limited to, antibodies to adhesion molecules and receptors of adhesion molecules in free forms. Herein, the free receptors of adhesion molecules include all receptors that are capable of specifically binding to adhesion molecules, and preferably are Ig fusion proteins in which receptors of adhesion molecules or soluble extracellular domains thereof are linked to immunoglobulins or Fc liagments thereof. Further, the Ig fusion proteins may be in additionally glycosylated forms.

The “cytokines” include IL-1, IL-2, IL-3, IL4, IL-5, IL-6, IL-7, TNF, TGF, IFN, GM-CSF, G-CSF, EPO, TPO and M-CSF, and their receptors include IL-1R, IL-2R, IL-3R, IL-4R, IL-5R, IL-6R, IL-7R, TNFR, TGFR, IFNR (e.g., IFN-γR α-chain, IFN-γR β-chain), IFN-αR, -β R and -γ R, GM-CSFR, G-CSFR, EPOR, cMp1 and gp130, respectively. Cytokines bind to their receptors on B lymphocytes or T lymphocytes and induce immune responses. Thus, immune responses induced by cytokines may be suppressed by blocking the binding between cytokines and their receptors. Substances capable of displaying such suppressive action include, but are not limited to, antibodies to cytokines and receptors of cytokines in free forms. Herein, the free cytokine receptors include all receptors that are capable of specifically binding to cytokines, and preferably are Ig fusion proteins in which cytokine receptors or soluble extracellular domains thereof are linked to immunoglobulins or Fc fragments thereof. Further, the Ig fusion proteins may be in additionally glycosylated forms.

I. Antibodies

Substances capable of blocking the binding of MHC Class II molecules and receptors thereof may include antibodies to MHC Class II molecules. Substances capable of blocking the binding of costimulatory molecules and receptors thereof may include antibodies to costimulatory molecules. Substances capable of blocking the binding of adhesion molecules and receptors thereof may include antibodies to adhesion molecules. Substances capable of blocking the binding of cytokines and receptors thereof may include antibodies to cytokines.

The antibodies may be polyclonal or monoclonal. Polyclonal and monoclonal antibodies may be commercially available or produced according to methods known in the art. A polyclonal antibody is generally produced by immunizing a mammal with a suitable amount of an antigen one or more times and recovering anti-sera from the immunized mammal when antibody titers reach desired levels. If desired, the anti-sera may be purified using a known process and stored in a frozen buffer solution until use. On the other hand, a monoclonal antibody may be prepared by injecting an antigen into a mammal, isolating generated B lymphocytes, fusing the B lymphocytes with myeloma cells and culturing the thus obtained hybridoma cells. Details of these processes are well known in the art.

II. Ig Fusion Proteins

Substances capable of blocking the binding of MHC Class II molecules and receptors thereof may include Ig fusion proteins with receptors of MHC Class II molecules. Substances capable of blocking the binding of costimulatory molecules and receptors thereof may include Ig fusion proteins with receptors of costimulatory molecules. Substances capable of blocking the binding of adhesion molecules and receptors thereof may include Ig fusion proteins with receptors of adhesion molecules. Substances capable of blocking the binding of cytokines and receptors thereof may include Ig fusion proteins with cytokine receptors. Hereinafter, receptors of MHC Class II molecules, receptors of costimulatory molecules, receptors of adhesion molecules and cytokine receptors are collectively called “receptors”.

The term “Ig fusion protein”, as used herein, refers to a fusion protein that includes a receptor protein or a soluble extracellular domain thereof linked to an immunoglobulin or an Fc fragment thereof. In detail, the Ig fusion protein includes simple fusion monomeric forms, simple fusion dimeric forms, concatameric fusion monomeric forms, concatameric fusion dimeric forms, and glycosylated forms thereof.

The term “soluble extracellular domain”, as used herein, refers to a portion exposed to the extracellular region of an integral membrane protein penetrating the cell membrane comprising phospholipid, wherein the integral membrane protein contains one or more transmembrane domain made up predominantly of hydrophobic amino acids. Such an extracellular domain mainly comprises hydrophilic amino acids, which are typically positioned at the surface of a folded structure of a protein, and thus is soluble in an aqueous environment. For most cell surface receptor proteins, extracellular domains serve to bind specific ligands, while intracellular domains play an important role in signal transduction.

The term “immunoglobulin”, as used herein, refers to protein molecules being produced in B cells and serving as antigen receptors specifically recognizing a wide variety of antigens. The molecules have a Y-shaped structure consisting of two identical light chains (L chains) and two identical heavy chains (H chains), in which the four chains are held together by a number of disulfide bonds, including the disulfide bridge between the H chains at the hinge region. The L and H chains comprise variable and constant regions. The L chain variable region associates with the H chain variable region, thus producing two identical antigen-binding regions. According to features of the constant regions of H chains, immunoglobulins (Ig) are classified into five isotypes, A (IgA), D (IgD), E (IgE), G (IgG) and M (IgM). Biological functions of immunoglobulin molecules, such as complement activation, Fc receptor-mediated phagocytosis and antigen-dependent cytotoxicity, are mediated by structural determinants (complementarity-determining regions) in the Fc region of H chains. Such an Fc region of H chains is used for construction of dimeric proteins according to the present invention, and may be derived from all isotypes of immunoglobulin as described above.

The term “Fc fragment of an immunoglobulin molecule”, as used herein, refers to a fragment having no antigen-binding activity and being easily crystallized, which comprises a hinge region and CH2 and CH3 domains, and a portion responsible for binding of an antibody to effector materials and cells.

The term “concatameric fusion”, as used herein, refers to a state in which the N-terminus of a soluble extracellular domain of a receptor protein is linked to the C-terminus of a soluble extracellular domain of the receptor protein, and thus two soluble extracellular domains of the receptor protein form a long polypeptide.

The term “simple fusion monomeric protein”, as used herein, refers to a fusion protein having a monomeric structure consisting of a single polypeptide formed by linkage of a soluble extracellular domain of a receptor protein to the hinge region of an Fc fragment of an immunoglobulin molecule. A simple fusion monomeric protein may be designated “receptor protein name/Fc” for convenience in the present invention. For example, a simple fusion monomeric protein produced by linkage of a soluble extracelllar domain of LAG3 protein to an Fc fragment of an immunoglobulin molecule is designated LAG3/Fc. If desired, the origin of the Fc fragment may be also specified in the designation. For example, in the case that the Fc fragment is derived from IgG1, the monomeric protein is called LAG3/IgG1Fc.

The term “simple fusion dimeric protein”, as used herein, refers to a fusion protein having a dimeric structure, in which two simple fusion monomeric proteins are joined by formation of intermolecular disulfide bonds at the hinge region. Such a simple fusion dimeric protein may be designated “[receptor protein name/Fc]₂” for convenience in the present invention. For example, when fused by formation of intermolecular disulfide bonds at the hinge region of two simple fusion monomeric proteins produced by linkage of an soluble extracellular domain of LAG3 protein and an Fc fragment of an immunoglobulin molecule, the resulting fusion protein having dimeric structure is designated [LAG3/Fc]₂. In addition, the origin of the Fc fragment may be specified in the designation, if desired. For example, in the case that the Fc fragment is derived from IgG1, the dimeric protein is designated [LAG3/IgG1Fc]₂.

The term “concatameric fusion monomeric protein”, as used herein, refers to a fusion protein having a monomeric structure consisting of a single polypeptide, in which the N-terminus of a soluble extracellular domain of a receptor protein is linked to the C-terminus of a soluble extracellular domain of the receptor protein, wherein the C-terminus of the former soluble extracellular domain is linked to the hinge region of an Fc fragment of an immunoglobulin molecule. A concatameric fusion monomeric protein may be designated “receptor protein name-receptor protein name/Fc” for convenience in the present invention. For example, when a soluble extracellular domain of LAG3 of a simple fusion monomeric protein, produced by linkage of the soluble extracellular domain of LAG3 protein and an Fc fragment of an immunoglobulin molecule, is linked to a soluble extracellular domain of LAG3, the resulting concatameric fusion monomeric protein is designated LAG3-LAG3/Fc. If desired, the origin of the Fc fragment may be specified in the designation. For example, in the case that the Fc fragment is derived from IgG1, the monomeric protein is designated LAG3-LAG3/IgG1Fc.

The term “concatameric fusion dimeric protein”, as used herein, refers to a fusion protein having a dimeric structure, in which two concatameric fusion monomeric proteins are fused by formation of intermolecular disulfide bonds at the hinge region. A concatameric fusion dimeric protein may be designated “[receptor protein name-receptor protein name/Fc]₂” for convenience in the present invention. For example, when two concatameric fusion monomeric proteins, each of which is produced by linkage of a LAG3 soluble extracellular domain of a simple fusion monomeric protein to a soluble extracellular domain of LAG3 protein, are fused by formation of intermolecular disulfide bonds at the hinge region, the resulting fusion protein having dimeric structure is designated [LAG3-LAG3/Fc]₂, wherein the simple fusion monomeric protein is formed by linkage of the LAG3 soluble extracellular domain to an Fc fragment from an immunoglobulin molecule. If desired, the origin of the Fc fragment may be specified in the designation. For example, in the case that the Fc fragment is derived from IgG1, the fusion protein is designated [LAG31-LAG3/IgG1Fc]₂.

On the other hand, a simple fusion monomeric protein or a simple fusion dimeric protein may be prepared according to a typical method known in the art. A concatameric fusion monomeric protein or a concatameric fusion dimeric protein may be obtained using a preparation method described in PCT Publication No. WO 2003/010202, which was filed by the present inventors.

The concatameric fusion dimeric protein according to the present invention is generally prepared by (a) preparing a DNA construct encoding a simple fusion monomeric protein using a gene encoding an Fc fragment of an immunoglobulin molecule and a gene encoding a soluble extracellular domain of a receptor protein; (b) inserting by polymerase chain reaction (PCR) a recognition sequence of a restriction enzyme into the prepared simple fusion monomeric protein-encoding DNA construct and the gene encoding a soluble extracellular domain of a receptor protein, respectively; (c) cleaving the recognition sequence of a restriction enzyme in the simple fusion monomeric protein-coding DNA construct and the gene encoding a soluble extracellular domain of a receptor protein using the restriction enzyme recognizing the recognition sequence; (d) ligating the cleaved DNA fragments using ligase to produce a DNA construct encoding a concatameric fusion monomeric protein; (e) operably linking the prepared DNA construct encoding a concatameric fusion monomeric protein to a vector to produce a recombinant expression plasmid; (f) transforming or transfecting a host cell with the recombinant expression plasmid; and (g) culturing the transformant or transfectant under conditions suitable for expression of the DNA construct encoding a concatameric fusion monomeric protein and then isolating and purifying a concatameric fusion dimeric protein of interest.

In accordance with the present invention, to allow additional O-linked or N-linked glycosylation, one or more nucleotides in a DNA sequence encoding a soluble extracellular domain of a receptor protein are altered, and the resulting DNA is expressed in a suitable animal host cell to induce glycosylation using the host system. In accordance with an aspect of the present invention, the glycosylated concatameric fusion dimeric protein according to the present invention may be prepared by altering a DNA sequence encoding a soluble extracellular domain of a receptor protein to induce or increase N-linked glycosylation by adding the sequence Asn-X-Ser/Thr.

The present invention will be described in detail with MHC Class II molecules, as well as B7 molecule as an illustrative example of the costimulatory molecule, LFA-3 molecule as an illustrative example of the adhesion molecule and TNF as an illustrative example of the cytokine.

The “MHC Class II molecules” are recognized by CD4 and LAG3 receptors, which are capable of specifically binding to MHC Class II molecules. Thus, an Ig fusion protein of LAG3 may be used for blocking the binding of MHC Class II molecules and CD4. In detail, substances capable of blocking the binding of MHC Class II molecules and CD4 include (1) an antibody to MHC Class II molecules; (2) a simple fusion monomeric protein formed by linkage of a soluble extracellular domain of LAG3 to the hinge region of an Fc fragment of an immunoglobulin molecule; (3) a simple fusion dimeric protein in which two molecules of the simple fusion monomeric protein are joined by intermolecular disulfide bonds in the hinge region; (4) a concatameric fusion monomeric protein formed by linkage of the N-terminus of a soluble extracellular domain of LAG3, linked to the hinge region of the simple fusion monomeric protein, to the C-terminus of a soluble extracellular domain of another LAG3 molecule; (5) a concatameric fusion dimeric protein in which two molecules of the concatameric fusion monomeric protein are joined by intermolecular disulfide bonds in the hinge region; and (6) glycosylated forms of the proteins according to (2) to (5).

The “B7 molecule” is recognize by CD28 and CTLA4, which are capable of specifically binding to the B7 molecule. In particular, the B7 molecule binds to CD28 expressed on the surface of T lymphocytes and activates T lymphocytes. In contrast, the B7 molecule suppresses the activation of T lymphocytes when binding to another receptor CTLA4 (expressed after T lymphocytes are activated). Thus, an Ig fusion protein of CTLA4 may be preferably used for blocking the binding of the B7 molecule and CD28. In detail, substances capable of blocking the binding of the B7 molecule and CD28 include (1) an antibody to the B7 molecule; (2) a simple fusion monomeric protein formed by linkage of a soluble extracellular domain of CTLA4 to the hinge region of an Fc fragment of an immunoglobulin molecule; (3) a simple fusion dimeric protein in which two molecules of the simple fusion monomeric protein are joined by intermolecular disulfide bonds in the hinge region; (4) a concatameric fusion monomeric protein formed by linkage of the N-terminus of a soluble extracellar domain of CTLA4, linked to the hinge region of the simple fusion monomeric protein, to the C-terminus of a soluble extracellular domain of another CTLA4 molecule; (5) a concatameric fusion dimeric protein in which two molecules of the concatameric fusion monomeric protein are joined by intermolecular disulfide bonds in the hinge region; and (6) glycosylated forms of the proteins according to (2) to (5).

The T lymphocyte-activating function of the “LFA3 molecule” may be suppressed by blocking the binding of LFA-3 and CD2 on the surface of T lymphocytes. Such immunosuppressive substances include (1) an antibody to LFA-3; (2) a simple fusion monomeric protein formed by linkage of a soluble extacellular domain of CD2 to the hinge region of an Fc fragment of an immunoglobulin molecule; (3) a simple fusion dimeric protein in which to molecules of the simple fusion monomeric protein are joined by intermolecular disulfide bonds in the hinge region; (4) a concatameric fusion monomeric protein formed by linkage of the N-terminus of a soluble extacellar domain of CD2, linked to the hinge region of the simple fusion monomeric protein, to the C-terminus of a soluble extracellular domain of another CD2 molecule; (5) a concatameric fusion dimeric protein in which two molecules of the concatameric fusion monomeric protein are joined by intermolecular disulfide bonds in the hinge region, and (6) glycosylated forms of the proteins according to (2) to (5).

The immune response-activating function of “TNF” may be suppressed by blocking the binding of TNF and TNFR on the surface of T lymphocytes. Such immunosuppressive substances include (1) an antibody to TNF; (2) a simple fusion monomeric protein formed by linkage of a soluble extracellular domain of TNFR to the hinge region of an Fc fragment of an immnunoglobulin molecule; (3) a simple fusion dimeric protein in which two molecules of the simple fusion monomeric protein are joined by intermolecular disulfide bonds in the hinge region; (4) a concatameric fusion monomeric protein formed by linkage of the N terminus of a soluble extracellar domain of TNFR, linked to the hinge region of the simple fusion monomeric protein, to the C-terminus of a soluble extracellular domain of another TNFR molecule; (5) a concatameric fusion dimeric protein in which two molecules of the concatameric fusion monomeric protein are joined by intermolecular disulfide bonds in the hinge region; and (6) glycosylated forms of the proteins according to (2) to (5).

III. Immunological Disorders

The active ingredients according to the present invention may be used for treating diverse diseases caused due to unwanted activation of T lymphocytes since they are able to suppress the activation of T lymphocytes. Representative examples of such diseases are transplantation rejection and autoimmune diseases.

“Transplantation rejection” refers to immune responses caused by the difference in genetic background between a donor of a graft (a part of a living body that is transplanted, a cell, a tissue, or an organ) and a recipient, and includes (1) a disease called “graft-versus-host disease (GVHD)”, which is caused when immune cells derived from a graft of a donor recognize a recipient as a foreign substance and attack the recipient, and (2) a disease called “graft rejection”, which is caused when a recipient recognizes a graft of a donor as a foreign substance and attacks the graft.

On the other hand, diseases occurring when immune cells do not distinguish between the self and the non-self (foreign) materials and attack the self are collectively called “autoimmune diseases”. In detail, autoimmune diseases include rheumatoid arthritis, multiple sclerosis, myasthenia gravis, Grave's disease, Hashimoto's thyroiditis, Addison's disease, vitilligo, scleroderma, Goodpasture syndrome, Becet's disease, Crohn's disease, ankylosing spondylitis, uveitis, thrombocytopenic purpura, pemphigus vulgaris, childhood diabetes, autoimmune anemia, cryoglobulinemia, adrenoleukodystrophy (ALD), and systemic lupus erythematosus (SLE).

IV. Pharmaceutical Composition

The pharmaceutical composition of the present invention may be preferably in a form such that therapeutically effective amounts of two or more active ingredients, selected from the group consisting of a substance capable of blocking binding of an MHC Class II molecule and a receptor thereof, a substance capable of blocking binding of a costimulatory molecule and a receptor thereof, a substance capable of blocking binding of an adhesion molecule and a receptor thereof, and a substance capable of blocking binding of a cytokine and a receptor thereof, are loaded in a pharmaceutically acceptable carrier.

The carrier used in the pharmaceutical composition of the present invention includes the commonly used carriers, adjuvants and vehicles, in the pharmaceutical field, which are as a whole called “pharmaceutically acceptable carriers”. Non-limiting pharmaceutically acceptable carriers useful in the pharmaceutical composition of the present invention include ion exchange, alumina, aluminum stearate, lecithin, serum proteins (e.g., human serum albumin), buffering agents (e.g., sodium phosphate, glycine, sorbic acid, potassium sorbate, partial glyceride mixtures of vegetable saturated fatty acids), water, salts or electrolytes (e.g., protamine sulfate, disodium hydrophosphate, potassium hydrophoshate, sodium chloride, and zinc salts), colloidal silica, magnesium trisilicate, polyvinylpyrrolidone, cellulose-based substrates, polyethylene glycol, sodium carboxymethylcellulose, polyarylate, waxes, polyethylene-polyoxypropylene-block copolymers, polyethylene glycol, and wool fat.

The pharmaceutical composition of the present invention may be administered via any of the common routes, if it is able to reach a desired tissue. Therefore, the pharmaceutical composition of the present invention may be administered topically, orally, parenterally, intraocularly, transdermally, intrarectally and intraluminally, and may be formulated into solutions, suspensions, tablets, pills, capsules and sustained release preparations. The term “parenteral”, as used herein, includes subcutaneous, intranasal, intravenous, intraperitoneal, intramuscular, intra-articular, intra-synovial, intrasternal, intracardial, intrathecal, intralesional and intracranial injection or infusion techniques.

In an aspect, the pharmaceutical composition of the present invention may be formulated as aqueous solutions for parenteral administration. Preferably, a suitable buffer solution, such as Hank's solution, Ringer's solution or physiologically buffered saline, may be employed. Aqueous injection suspensions may be supplemented with substances capable of increasing viscosity of the suspensions, which are exemplified by sodium carboxymethylcellulose, sorbitol and dextran. In addition, suspensions of the active ingredients, such as oily injection suspension, include lipophilic solvents or carriers, which are exemplified by fatty oils such as sesame oil, and synthetic fatty acid esters such as ethyl oleate, triglycerides or liposomes. Polycationic non-lipid amino polymers may also be used as vehicles. Optionally, the suspensions may contain suitable stabilizers or drugs to increase the solubility of protein variants and obtain high concentrations of the protein variants.

The pharmaceutical composition of the present invention is preferably in the form of a sterile injectable preparation, such as a sterile injectable aqueous or oleaginous suspension. Such suspension may be formulated according to the methods known in the art, using suitable dispersing or wetting agents (e.g., Tween 80) and suspending agents. The sterile injectable preparations may also be a sterile injectable solution or suspension in a non-toxic paternally-acceptable diluent or solvent, such as a solution in 1,3-butanediol. The acceptable vehicles and solvents include mannitol, water, Ringer's solution and isotonic sodium chloride solution. In addition, sterile fixed oils may conventionally be employed as a solvent or suspending medium. For this purpose, any bland fixed oil may be employed, including synthetic mono- or di-glycerides. In addition, fatty acids, such as oleic acid and glyceride derivatives thereof, may be used in the preparation of injectable preparations, like the pharmaceutically acceptable natural oils (e.g., olive oil or castor oil), and particularly, polyoxyethylated derivatives thereof.

The aforementioned aqueous composition is sterilized only by filtration using a filter to remove bacteria, mixing with disinfectants or in combination with radiation. The sterilized composition can be hardened, for example, by freeze-drying to obtain a hardened product, and for practical use, the hardened product is dissolved in sterilized water or a sterilized diluted solution.

In order to increase stability at room temperature, reduce the need for high-cost storage at low temperature, and prolong shelf-life, the pharmaceutical composition comprising active ingredients according to the present invention may be lyophilized. A process for freeze-drying may comprise the steps of freezing, first drying and second drying. After freezing, the composition is heated under pressure to evaporate vapor. At the second drying step, residual water is removed from the dry product.

The term “therapeutically effective amount”, as used herein in connection with the pharmaceutical composition of the present invention, means an amount in which active ingredients show an improved or therapeutic effect toward a immunological disease to which the pharmaceutical composition of the present invention is applied. The therapeutically effective amount of the pharmaceutical composition of the present invention may vary according to the patient's age and sex, application sites, administration frequency, administration duration, formulation types and adjuvant types. Typically, the pharmaceutical composition of the present invention is administered in amounts, for example, 0.01-1000 μg/kg/day, more preferably 0.1-500 μg/kg/day, and most preferably 1-100 μg/kg/day.

The present invention will be explained in more detail with reference to the following examples in conjunction with the accompanying drawings. However, the following examples are provided only to illustrate the present invention, and the present invention is not limited to them.

The following Example 1 relates to LAG3. Information on amino acid sequences of LAG3/Fc and LAG3-LAG3/Fc fusion proteins, DNA sequences encoding the fusion proteins and primers used for preparing the fusion proteins is summarized in Table 1, below 1.

TABLE 1
Information on DNA and amino acid sequences
of LAG3/Fc and LAG3-LAG3/Fc and primers used
for preparing the fusion proteins

| | SEQ ID | |
|---|---|---|
| | No. | Remarks |
| Oligo-LAG3-F-EcoRI | 1 | Primer containing the 5′-end |
| | | of a soluble extracellular |
| | | domain of LAG3 and an EcoRI site |
| Oligo-LAG3-R-5P | 2 | Primer containing the 3′-end |
| | | of a soluble extracellular |
| | | domain of LAG3 |
| Oligo-LAG3-F-5P | 3 | Primer containing the 5′-end |
| | | of a soluble extracellular |
| | | domain of LAG3 |
| Oligo-LAG3-R-SpeI | 4 | Primer containing the 3′-end |
| | | of a soluble extracellular |
| | | domain of LAG3 and |
| | | an SpeI site |
| hIgG-F-SpeI | 5 | Primer containing the 5′-end |
| | | of an IgG hinge region and an |
| | | SpeI site |
| hIgG-R-XbaI | 6 | Primer containing the 3′-end |
| | | of IgG and an XbaI site |
| DNA sequence encoding | 7 | — |
| LAG3/FC | | |
| Amino acid sequence of | 8 | — |
| LAG3/Fc | | |
| DNA sequence encoding | 9 | — |
| LAG3-LAG3/Fc | | |
| Amino acid sequence of | 10 | — |
| LAG3-LAG3/FC | | |

### EXAMPLE 1

### Preparation of DNA Constructs Encoding Ig Fusion Proteins According to the Present Invention

A. Manufacture of a DNA Construct Encoding Simple Fusion Monomeric Protein of LAG3/Fc

a. DNA fragment encoding soluble extracellular domain of LAG3

A DNA fragment encoding soluble extracellular domain of LAG3 was constructed by PCR using a primer (the sequence of nucleotide of SEQ ID NO: 1) with EcoRI restriction site and the sequence (the sequence of nucleotide of SEQ ID NO: 7) encoding leader sequence (the sequence of amino acids 1-22 of SEQ ID NO: 8), and an antisense primer (the sequence of nucleotide of SEQ ID NO: 4) with SpeI restriction site and the sequence (the sequence of nucleotide of SEQ ID NO: 7) encoding a part of 3′ ends of the said soluble extracellular domain of LAG3. The template cDNA for this reaction was constructed by reverse transcription PCR (RT-PCR) of mRNA extracted from monocyte (T lymphocyte) of healthy adults.

After blood of healthy adults was extracted and diluted to 1:1 with RPMI-1640 (Gibco BRL, USA), the layer of T lymphocyte which formed at upper part was obtained by density gradient centrifugation using Ficoll-hypaque (Amersham, USA). The cell was washed with RPMI-1640 for 3 times, and RPMI-1640 culture media containing 10% Fetal Bovine Serum (FBS, Gibco BRL, USA) was added to make the concentration of the cell to 5×10⁵ cell/ml then stimulated after adding phytohemagglutinin-M(Calbiochem, Germany) to 2 μg/ml.

The mRNAs were purified using Tri-Reagent (MRC, USA) mRNA purification kit. First, 2×10⁷ of human T lymphocyte was washed with Phosphate Buffered Saline (PBS, pH7.2) for 3 times, and then 1 ml of Tri-Reagent was mixed for several times to dissolve RNA. After adding 0.2 of chloroform to this tube and mixing thoroughly, this tube was incubated at room temperature (RT) for 15 min, then centrifuged at 15,000 rpm, 4° C. for 15 min. The upper part of the solution was transferred to a 1.5ml tube, and 0.5 ml of isopropanol was added, and then centrifuged at 15,000 rpm, 4° C. for 15 min. After the supernatant was discarded, the pellet was resuspended with 1 ml of 3° C. distilled water treated with 75% ethanol-25% DEPC (Sigma, USA), and then centrifuged at 15,000 rpm, 4° C. for 15 min. After the supernatant was removed completely and dried in the air to remove ethanol residue, RNA was resuspended with 50 μl of 3° distilled water treated with DEPC.

The primary cDNA was synthesized by mixing 2 μg of purified mRNA and 1 μl of oligo dT (dT30, Promega, USA) primer to 10 μM in 1.5 ml tube, heating at 70° C. for 2 min, and cooling in ice for 2 min. After that, this mixture was added with 200 U of M-MLV reverse transcriptase (Promega, USA), 10 μl of 5× reaction buffer (250 mM Tris-HCl, pH 8.3, 375 mM KCl, 15 mM MgCl₂, and 50 mM DTT), 1 μl of dNTP (10 mM each, Takara, Japan), and DEPC-treated 3° distilled water to 50 μl, then reacted at 42° C. for 1 hour.

b. DNA fragment encoding Fc fragment of immunoglobulin GI

A DNA fragment encoding Fc fragment of immunoglobulin GI was constructed by PCR using a primer (the sequence of nucleotide of SEQ ID NO: 5) with SpeI restriction site and the sequence encoding a part of 5′ end of the hinge region of immunoglobulin GI (IgG1), and an antisense primer (the sequence of nucleotide of SEQ ID NO: 6) with Xbal restriction site and the sequence encoding 3′ ends of IgG1 Fc. The template cDNA for this reaction was constructed by RT-PCR of mRNA extracted from peripheral blood cell (B lymphocyte) of convalescent patients with pyrexia of unknown origin.

c. DNA construct encoding simple fusion monomeric protein of LAG3/Fc

Both of DNA fragment encoding soluble extracellular domain of LAG and DNA fragment encoding Fc fragment of immunoglobulin produced as described above were restricted with SpeI and ligated using T₄ ligase(USB, USA), thus producing simple fusion monomeric protein of LAG/Fc.

d. Cloning of the DNA construct encoding simple fusion monomeric protein of LAG/Fc

DNA construct encoding simple fusion monomeric protein of LAG/Fc as described above was restricted with EcoRI and XbaI, and cloned by inserting into a commercially available cloning vector, pBluescript KS II (+) (Stratagene, USA), at EcoRI/XbaI site. The sequence of a total coding region was identified by DNA sequencing (SEQ ID NO: 7). This produced fusion protein was designated LAG3/Fc as simple fusion monomeric protein, and the deduced amino acid sequence of simple fusion monomeric of LAG3/Fc corresponded to SEQ ID NO: 8.

B. Manufacture of a DNA construct encoding concatameric fusion monomeric protein of LAG3-LAG3/Fc

In order to produce a DNA construct encoding concatameric fusion monomeric protein of LAG3-LAG3/Fc, a DNA fragment encoding soluble extracellular domain of LAG3 was constructed by PCR using a primer (the sequence of nucleotide of SEQ ID NO: 1) with EcoRI restriction site and the sequence (the sequence of nucleotide of SEQ ID NO: 7) encoding leader sequence (the sequence of amino acids 1-22 of SEQ ID NO: 8), and an antisense primer (the sequence of nucleotide of SEQ ID NO: 4) with the sequence (the sequence of nucleotide of SEQ ID NO: 7) encoding a part of 3′ ends of the said soluble extracellular domain of LAG3. Also, a DNA fragment encoding simple fusion monomeric protein of LAG3/Fc was constructed by PCR using a primer (the sequence of nucleotide of SEQ ID NO: 3) encoding termination parts (the sequence of nucleotide of SEQ ID NO: 7) of leader sequence of soluble extracellular domain of LAG3 and an antisense primer (the sequence of nucleotide of SEQ ID NO: 6) with XbaI restriction site and the sequence encoding 3′ ends of IgG1 Fc. For these PCR, a DNA fragment encoding simple fusion monomeric protein of LAG3/Fc(the sequence of nucleotide of SEQ ID NO: 7) was used as the template.

PCR was performed by adding 1 μl of primary cDNA, 2 U of Pfu DNA polymerase (Stratagene, USA), 10 μl of 10 × reaction buffer [200 mM Tris-HCl, pH 8.75, 100 mM (NH₄)₂SO_{4,} 100 mM KCl, 20 mM MgCl₂]1% Triton™ X-100, 1 mg/ml BSA, 3 μl primer 1 (10 μM), 3 μl primer 2 (10 μM), 2 μl dNTP (10 mM each), and 3° distilled water to 100 μl. The reaction condition was as follows; 94° C., 5 min; 95° C., 1 min; 58° C., 1 min 30 sec; 72° C., 1 min for 31 cycles; and 72° C., 15 min to make PCR product with complete blunt end.

After electrophorized on 0.8% agarose gel, the PCR product was purified by Qiaex II gel extraction kit (Qiagen, USA). The purified PCR product was restricted by BamHI and extracted by phenol-chloroform extraction methods. Subsequently, two kinds of DNA fragments restricted by BamHI were linked by ligase.

C. Cloning of DNA constructs encoding concatameric fusion monomeric protein of LAG3-LAG3/Fc

DNA construct encoding concatameric fusion monomeric protein of LAG3-LAG3/Fc as described above was restricted with EcoRI and XbaI, and cloned by inserting into a commercially available cloning vector, pBluescript KS II (+) (Stratagene, USA), at EcoRI/XbaI site. The sequence of a total coding region was identified by DNA sequencing (SEQ ID NO: 9). This produced fusion protein was designated LAG3-LAG3/Fc as concatameric fusion monomeric protein and its deduced amino acid sequence corresponded to SEQ ID NO: 10.

After 10 μg of pBluescript KS II (+) (Stratagene, USA) used as a vector was mixed with 15 U of EcoRI 15 U of XbaI, 5 μl of 10× reaction buffer (100 mM Tris-HCl pH 7.5, 100 mM MgCl₂, 10 mM DTT, 500 nM NaCl), 5 μl of 0.1% BSA (Takama, Japan), and 3° distilled water to 50 μl , DNA was restricted by incubation at 37° C. for 2 hrs. After electrophorized on 0.8% agarose gel, the PCR product was purified by Qiaex II gel extraction kit (Qiagen, USA).

After 100 ng of pBluescript KS II (+) (Stratagene, USA) restricted by EcoRI and XbaI was mixed with 20 ng of PCR product restricted by the restriction enzyme, 0.5 U of T4 DNA ligase (Amersham, USA), 1 μl of 10× reaction buffer (300 mM Tris-HCl, pH 7.8, 100 mM MgCl₂, 100 mM DTT, 10 mM ATP) and 3° distilled water were added to 10 μl, and the mixture was incubated in the water bath at 16° C. for 16 hrs.

E. coli Top10 (Novex, USA) was made to competent cell by the method of rubidium chloride (RbCl, Sigma, USA) and transformed with the plasmid as described above, then spread on the solid LB media including 50 μg/ml of ampicillin (Sigma, USA) and incubated at 37° C. for 16 hrs. Formed colonies were inoculated in 4 ml of liquid LB media including 50 μg/ml of ampicillin and incubated at 37° C. for 16 hrs. Plasmid was purified by the method of alkaline lysis according to Sambrook et al. (Molecular cloning, Cold Spring Harbor Laboratory press, p125-1.31, pl.63-1.69, p7.26-7.29, 1989) from 1.5 μl of that, and the existence of cloning was confirmed by the restriction of EcoRI and XbaI.

The sequence of a total coding region was identified by the DNA sequencing method of dideoxy chain termination method (Sanger et al., Proc. Natl. Acad. Sci., 74:5483, 1977) as follows. The DNA sequencing reaction was performed according to the manual using a plasmid purified by alkaline lysis method as described above and Sequenase™ ver 2.0 (Amersham, USA). After the reaction mixture as above was loaded on 6% polyacrylamide gel and electrophorized for 2 hrs at constant voltage of 1,800-2,000 V and 50° C., DNA sequence was identified by exposing to X-ray film (Kodak, USA) after the gel was dried out.

### EXAMPLE 2

### Preparation of DNA Constructs Encoding Ig Fusion Proteins According to the Present Invention

Simple fusion dimeric proteins and concatameric fusion dimeric proteins for other proteins, TNFR1, TNFR2, CD2 and CTLA4, were prepared according to the same procedure as in Example 1. The procedure is described in detail in PCT Publication No. WO 2003/010202, which was filed by the present inventors. Information on DNA and amino acid sequences of Ig fusion proteins of TNFR1, TNFR2, CD2 and CTLA4 is summarized in Table 2, below.

TABLE 2
Ig fusion proteins according to the present invention
and DNA and amino acid sequences thereof

| | SEQ ID No. |
|---|---|
| DNA sequence encoding TNFR2/Fc | 11 |
| Amino acid sequence of TNFR2/Fc | 12 |
| DNA sequence encoding TNFR2-TNFR2/Fc | 13 |
| Amino acid sequence of TNFR2-TNFR2/Fc | 14 |
| DNA sequence encoding CD2/Fc | 15 |
| Amino acid sequence of CD2/Fc | 16 |
| DNA sequence encoding CD2-CD2/Fc | 17 |
| Amino acid sequence of CD2-CD2/Fc | 18 |
| DNA sequence encoding CTLA4/Fc | 19 |
| Amino acid sequence of CTLA4/FC | 20 |
| DNA sequence encoding CTLA4-CTLA4/FC | 21 |
| Amino acid sequence of CTLA4-CTLA4/FC | 22 |
| DNA sequence encoding TNFR1/Fc | 23 |
| Amino acid sequence of TNFR1/Fc | 24 |
| DNA sequence encoding TNFR2-TNFR1/Fc | 25 |
| Amino acid sequence of TNFR2-TNFR1/Fc | 26 |

### EXAMPLE 3

### Expression and Purification of Simple/Concatameric Fusion Dimeric Protein of LAG3/Fc

In order to express the fusion proteins in CHO-K1 cell (ATCC CCL-61, Ovary, Chinese hamster, Cricetulus griseus), after pBluescript KS II (+) plasmid DNA including LAG3-LAG3/Fc fusion gene was purified from transformed E. coli, an animal cell expression vectors were constructed as LAG3-LAG3/Fc figment produced by restriction using EcoRI and XbaI was inserted at EcoRI/XbaI site of an animal cell expression vector, pCR™3 (Invitrogen, USA) plasmid. And these were designated plasmid pLAG3-Top10′, and deposited as accession numbers of KCCM-10556, at Korean Culture Center of Microorganisms (KCCM, 361-221, Yurim B/D, Hongje-1-dong, Seodaemun-gu, SEOUL 120-091, Republic of Korea) on Jan. 13, 2004.

Transfection was performed by mixing the plasmid pLAG331Ig DNA including LAG3-LAG3/Fc fusion genes as described above with the reagent of Lipofectamin™ (Gibco BRL, USA). CHO-K1 cells with the concentration of 1˜3×10⁵ cells/well were inoculated in 6-well tissue culture plate (Nunc, USA), and incubated to 50˜80% in 10% FBS-DMEM media. Then the DNA-liposome complex, which was reacted for 15˜45 min with 1˜2 μg of either the plasmid pLAG33Ig DNA including LAG3-LAG3/Fc fusion genes as described above and 2˜25 μl of Lipofectamin™ (Gibco BRL, USA), were added to the cell culture plate in the serum-free DMEM media. After incubation for 5 hrs, DMEM media with 20% serum was added and cells were incubated further for 18˜24 hrs. After primary transfection, cells were incubated for 3 weeks in 10% FBS-DMEM media with 1.5 μg/ml of Geneticin (G418, Gibco BRL, USA), and formed colonies was selected for amplified incubation. The expression of fusion proteins was analyzed by ELISA using a peroxidase labeled goat anti-human IgG (KPL, USA).

ELISA was performed as follows. First, 1 μg/ml of a peroxidase labeled goat anti-human IgG (KPL, USA) was diluted to 1:2,000 with 0.1M sodium bicarbonate, 100 μl of that was aliquoted into 96-well flexible plate (Falcon, USA) and sealed with plastic wrap, then incubated at 4° C. over 16 hrs to be coated on the surface of the plate. After this, it was washed for 3 times with washing buffer (0.1% Tween-20 in 1× PBS) and then dilution buffer (48.5 ml 1XPBS, 1.5 ml FBS, 50 μl Tween-20) was aliquoted to 180 μl. After 20 μl of culture supernatant was dropped in the first well, then serially diluted using a micropipette, and 0.01 μg/ul of human immunoglobulin G (Sigma, USA) as the positive control and the culture media of untransfected CHO K-1 cell as the negative control was equally diluted. After dilution, 96-well ELISA plate (Falcon, USA) was wrapped with aluminum foil and incubated at 37° C. for 1 hr 30 min, washed for 3 times with washing buffer. Peroxidase conjugated goat anti-human IgG (KPL, USA) was diluted to 1:5,000 with dilution buffer, aliquoted to 100 μl, wrapped with aluminum foil, and reacted at 37° C. for 1 hr. After reaction, this plate was washed for 3 times, colorized using TMB microwell peroxidase substrate system (KPL, USA) and existence of expression was confirmed by measurement of absorbance at 655 nm wavelength using microplate reader (Bio-Rad, Model 550, Japan).

Adaptation for transfectants as described above to one of the serum free media, CHO-S-SFM II (Gibco BRL, USA), was proceeded to purify the proteins produced by those transfectants as follows. After about 3×10⁵ of cells were inoculated into the 6-well plate, cells were cultured at 5% CO2, 37° C. for over 16 hrs to adhere, and it was checked under a microscope that cells were adhered at about 30˜50% area of the plate, then cells were cultured in a media consisting of 10% FBS DMEM and CHO-S-SFM II in the ratio of 8:2. After culturing 3 times serial passage at this ratio, it was cultured 3 times at the ratio of 6:4; 3 times at 4:6; 3 times at 3:7; 3 times at 2:8; 3 times at 1:9; and finally cultured in 100% CHO-S-SFM II media. And the level of expression was measured by ELISA.

After these transfectant cells were cultured on a large scale in CHO-S-SFM II, the supernatants including each fusion protein were centrifuged at 200× g for 12 min to remove cell debris, and proteins were purified by the method using HiTrap protein A column (Amersham, USA) as follows. After 20 mM of sodium phospate (pH 7.0, Sigma, USA) was passed at the velocity of 1 ml/min for 2 min, 10 ml of supernatant was passed at the same velocity to bind fusion protein to protein A. After 20 mM of sodium phosphate (pH 7.0) was passed at the same velocity for 2 min to wash, 500 μl of the extracts were serially fractionated in a 1.5 ml tube as 0.1M of citric acid (pH 3.0, Sigma, USA) was passed at the the same velocity for 3 min. This was adjusted to pH 7.0 using 1M of Tris (pH 11.0, USB, USA), the existence of fusion proteins in tube was confirmed through ELISA as described above. The purified proteins were concentrated by centrifugation at 2000× g, 4° C. for 30 min using Centricon 30 (Amicon, USA).

### EXAMPLE 4

### Expression and Purification of Simple/Concatameric Fusion Dimeric Proteins for CD2, CTLA4 and TNFR

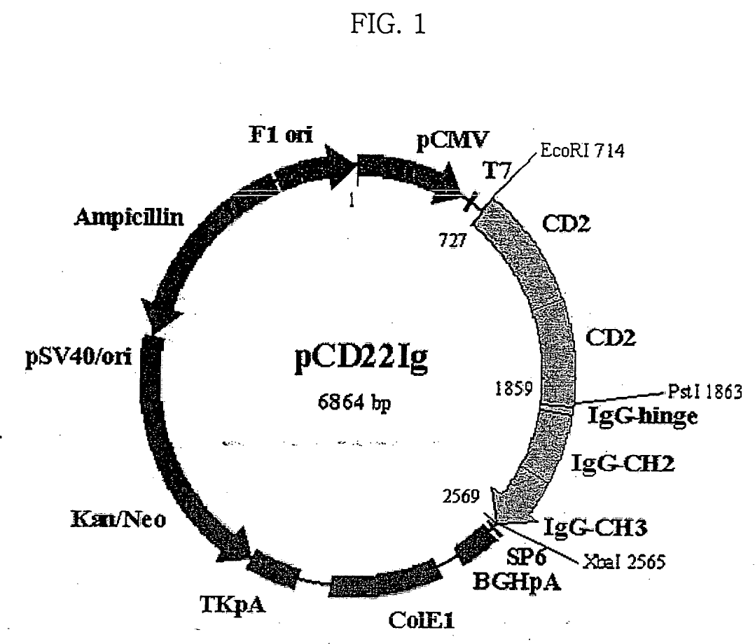

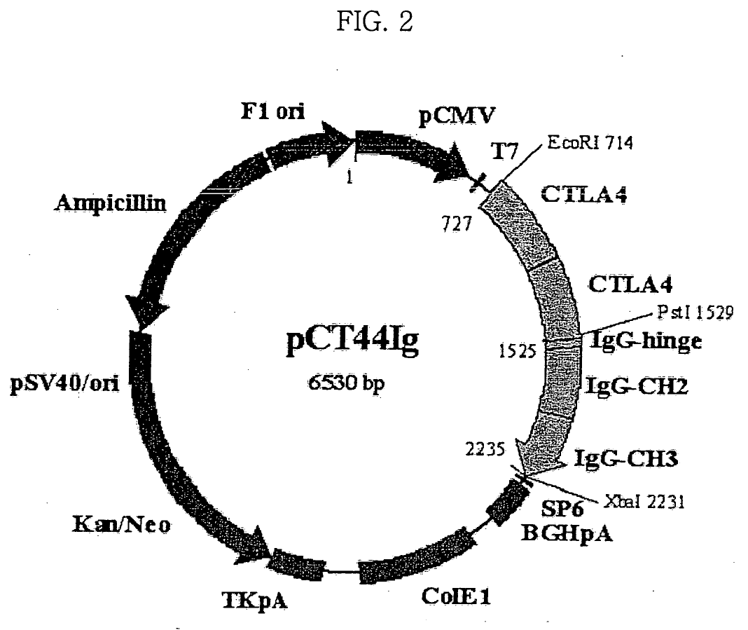

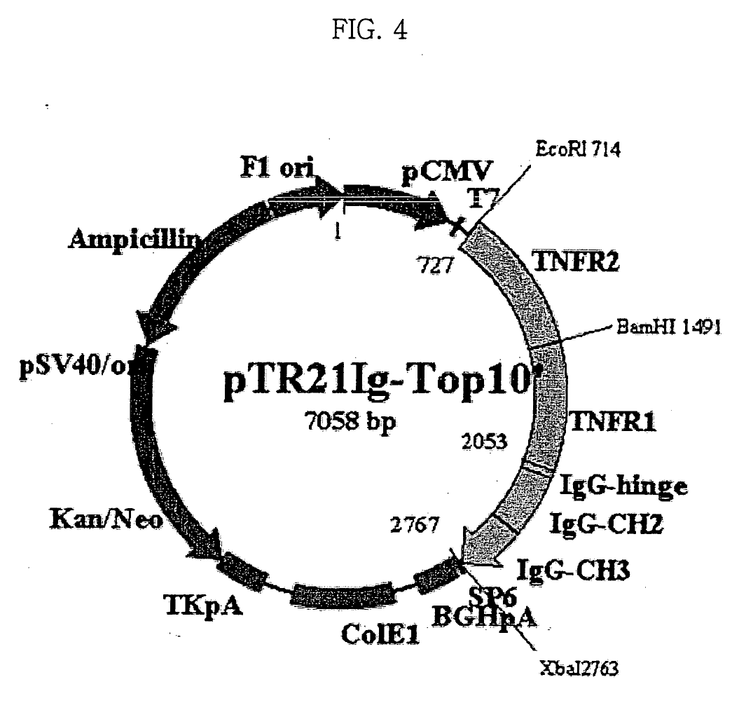

Simple/concatameric fusion dimeric proteins for CD2, CTLA4 and TNFR were prepared according to the same procedure as in Example 3. The procedure is described in detail in PCT Publication No. WO 2003/010202, which was filed by the present inventors. The thus obtained recombinant expression plasmids were designated pCD22Ig (FIG. 1), pCT44Ig (FIG. 2) and pTR21Ig-Top′ (FIG. 4), respectively.

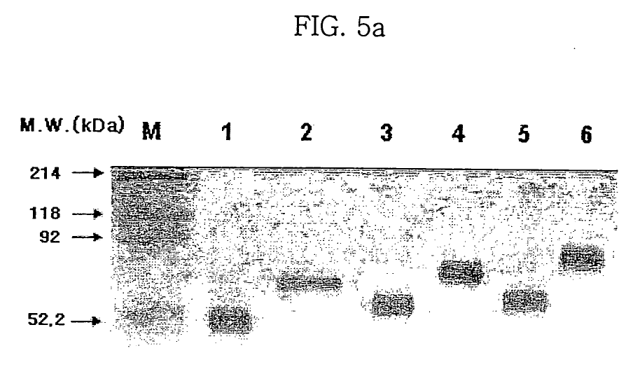

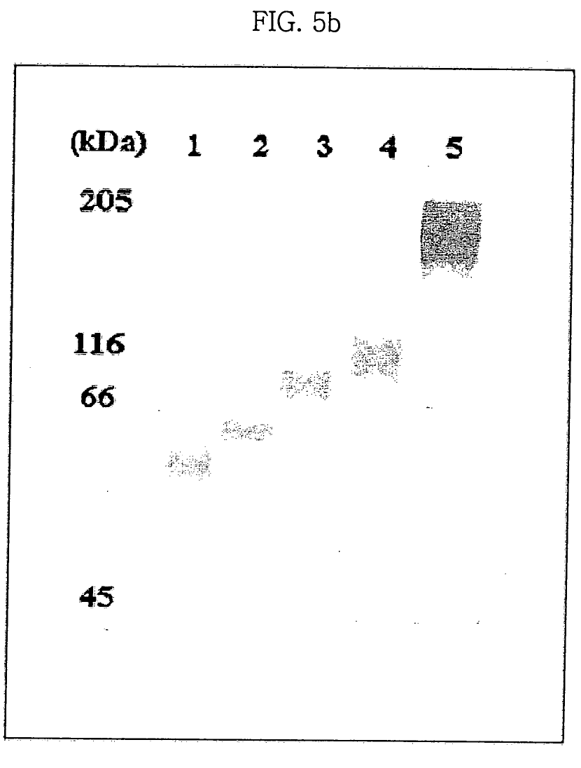

In addition, SDS-PAGE was performed to determine whether proteins purified in Examples 3 and 4 are desired simple fusion dimeric proteins [CD2/Fc]_{2,} [LAG3/Fc]₂ and [CTLA4/Fc]₂ and desired concatameric fusion dimeric proteins [CD2-CD2/Fc]₂, [LAG3-LAG3/Fc]₂ and [CTLA4-CTLA4/Fc]₂ (FIG. 5a). Also, SDS-PAGE was carried out for [TNFR1/Fc]₂, [TNFR2/Fc]_{2,} [TNFR2-TNFR1/Fc]₂ and [TNFR2-TNFR2/Fc]₂ (FIG. 5b).

### EXAMPLE 5

### Evaluation of the Inhibitory Effects of the Simple Fusion Dimeric Proteins or Concatameric Fusion Dimeric Proteins on T Lymphocyte Proliferation when the Proteins are used Separately or in Combination

A. The inhibitory effects of the simple fusion dimeric proteins on T lymphocyte proliferation when the proteins are used separately.

A B lymphocyte cell line, WT100B1S, which was prepared by transfecting B lymphocytes from febrile patients with Ebstein-Barr virus, was cultured in 10% fetal bovine serum (FBS)-containing RPMI 1640 to be used as antigen presenting cells for T lymphocytes. The cells were then centrifuged at 2,000 rpm for 2 min, and the cell pellet was suspended in 10% FBS-containing RPMI 1640 in a density of 5.0×10⁵ cells/ml and irradiated with γ-rays (3,000 rad).

T lymphocytes were isolated from blood samples collected from healthy people using Ficoll-Hypaque (Amersham, USA), and cultured in 10% FBS-containing RPMI 1640 to obtain a cell suspension of 2.0×10⁶ cells/ml.

A Primary Mixed Lymphocyte Reaction (MLR) was carried out as follows. 15 ml of the WT100B1S cell suspension was mixed with 15 ml of the suspension of T lymphocytes in a 150-mm culture dish. The cells were cultured for 3 days and further cultured for 3 days in 15 ml of 10% FBS-containing RPMI 1640. After the 6-day culture, viable T lymphocytes were isolated using Ficoll-Hypaque (Amersham, USA). The thus isolated T lymphocytes were frozen in a medium containing 45% FBS, 45% RPMI 1640 and 10% DMSO and stored in liquid nitrogen.

T lymphocytes from the primary MLR were rechallenged in a secondary MLR. First, the frozen T lymphocytes were thawed, washed with RPMI 1640 twice and resuspended in 10% FBS-containing RPMI 1640 at a density of 3×10⁵ cells/ml.

WT100B1S to be used as antigen presenting cells were newly cultured according to the aforementioned method. The cells were irradiated with γ-rays (3,000 rad) and suspended in 10% FBS-containing RPMI 1640 in a density of 7.5×10⁴ cells/ml. 100 μl of the WT100B1S cell suspension was plated onto each well of a 96-well flat-bottom plate, and the simple fusion dimeric proteins, [TNFR2/Fc]₂, [CD2/Fc]₂, [CTLA4/Fc]₂ and [LAG3/Fc]_{2,} were added to each well at final concentrations of 10, 1, 10⁻¹, 10⁻², 10⁻³ and 10⁻⁴ μg/ml. Then, 100 μl of T lymphocytes from the primary MLR were added to each well. The plate was incubated in a 5% CO2 incubator at 37° C. for 2 days, and 100 μl of 10% FBS-containing RPMI 1640 was added to each well, followed by further incubation for 2 days. For the last 6 hours during the 4-day culture, the cells were treated with 1.2 μCi/ml of ³H-thymidine (Amersham).

Thereafter, the 96-well plate was centrifuged at 110× g for 10 min at 4° C. to precipitate T lymphocytes. After the supernatants were discarded, the cell pellets were washed with 200 μl of 1× phosphate buffered saline (PBS). The plate was centrifuged under the same conditions to remove PBS. In order to eliminate remaining ³H-thymidine (Amersham), 200 μl of pre-cooled 10% trichloridic acid (TCA, Merck) was added to each well, and the plate was swirled for 2 min and allowed to react for 5 min at 4° C.

The plate was then centrifuged under the same conditions. After the supernatants were discarded, 200 μl of pre-cooled 70% ethanol was added to each well, and the plate was allowed to stand for 5 min at 4° C. to fix T lymphocytes. After the plate was centrifuged and the supernatants were discarded, the cells were treated with 10% TCA, and remaining ³H-thymidine (Amersham) was completely removed, according to the same method as described above.

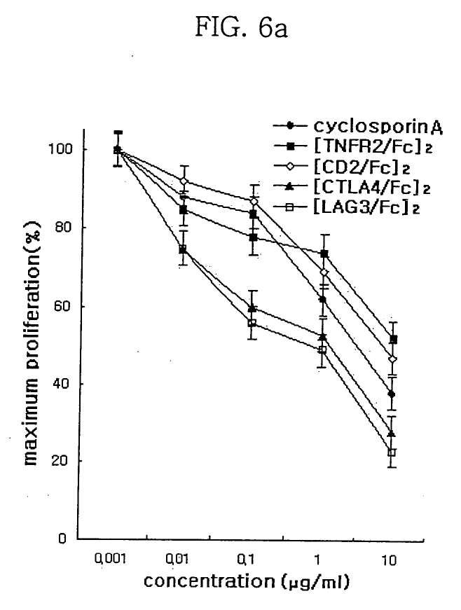

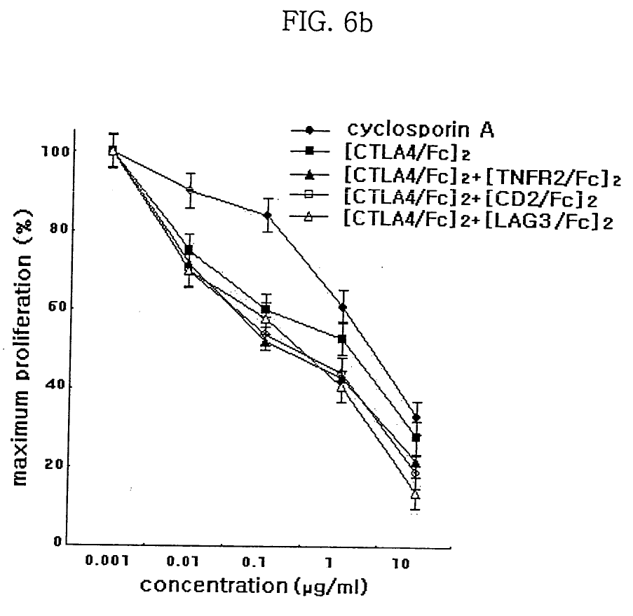

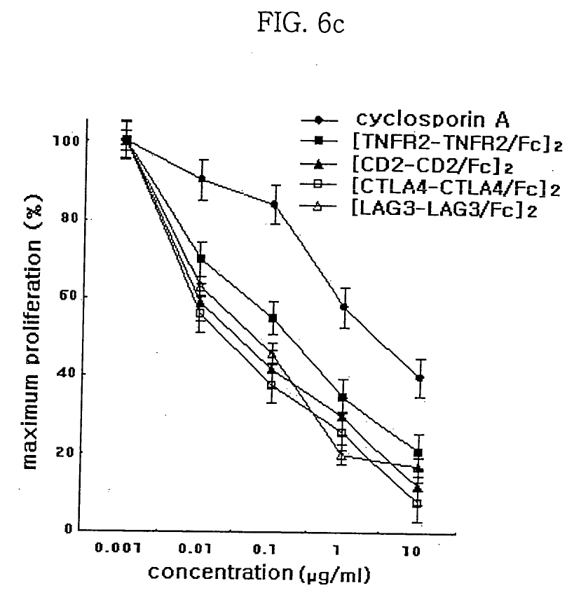

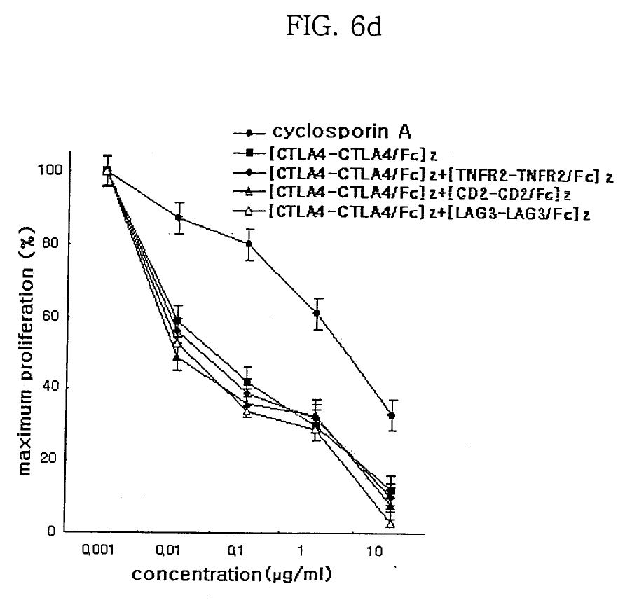

100 μl of 2% SDS (pH 8.0)/0.5N NaOH was then added to each well, and the plate was incubated for 30 min at 37° C. to lyse T lymphocytes. The plate was centrifuged at 110× g for 10 min at 25° C. to precipitate cell debris, and 50 μl of each supernatant was transferred to a 9-well sample plate (Wallac). 1.5 volumes of OptiPhase SuperMix (Wallac) were added to each well, and the plate was swirled for 5 min. The proliferation of T lymphocytes was determined by assessing the incorporation of ³H-thymidine through the measurement of radioactivity recorded as counts per minute (cpm) using a liquid scintillation counter (1450 MicroBeta TriLux microplate liquid scintillation and luminescence counter, Wallac) (FIG. 6a).

As shown in FIG. 6a, the simple fusion dimeric proteins [TNFR2/Fc]_{2,} [CD2/Fc]₂, [CTLA4/Fc]₂ and [LAG3/Fc]₂ all inhibited the proliferation of T lymphocytes. In particular, [CTLA4/Fc]₂ and [LAG3/Fc]₂ diplayed higher inhibitory effects on T lymphocyte proliferation than [TNFR2/Fc]₂ and [CD2/Fc]₂.

B. The inhibitory effects of the simple fusion dimeric proteins on T lymphocyte proliferation when the proteins are used in combination

The proliferation of T lymphocytes was assessed according to the same procedure as in the A of Example 5 except that the simple fusion dimeric proteins were used not separately but in combinations of [CTLA4/Fc]₂+[TNFR2/Fc]₂, [CTLA4/Fc]₂+[CD2/Fc]₂ and [CTLA4/Fc]₂+[LAG3/Fc]₂ along with [CTLA4/Fc]₂ alone as a control (FIG. 6b).

As shown in FIG. 6b, the combinations of [CTLA4/Fc]₂+[TNFR2/Fc]₂, [CTLA4/Fc]₂+[CD2/Fc]₂ and [CTLA4/Fc]₂+[LAG3/Fc]₂ as well as [CTLA4/Fc]₂ alone inhibted T lymphocyte proliferation. Also, the simple fusion dimeric proteins were found to be more effective in inhibiting the proliferation of T lymphocytes when used in combinations of two than when separately used.

C. The inhibitory effects of the concatameric fusion dimeric proteins on T lymphocyte proliferation when the proteins are used separately

The proliferation of T lymphocytes was assessed according to the same procedure as in the A of Example 5 except that, instead of the simple fusion dimeric proteins, the concatameric fusion dimeric proteins, [TNFR2-TNFR2/Fc]₂, [CD2-CD2/Fc]₂, [CTLA4-CTLA4/Fc]₂ and [LAG3-LAG3/Fc]₂, were used separately (FIG. 6c).

As shown in FIG. 6c, the concatameric fusion dimeric proteins [TNFR2-TNFR2/Fc]₂, [CD2-CD2/Fc]₂, [CTLA4-CTLA4/Fc]₂ and [LAG3-LAG3/Fc]₂ all inhibited the proliferation of T lymphocytes. Also, the concatameric fusion dimeric proteins used separately were found to have stronger inhibitory effects on T lymphocyte proliferation than the simple fusion dimeric proteins used separately.

D. The inhibitory effects of the concatameric fusion dimeric proteins on T lymphocyte proliferation when the proteins are used in combination

The proliferation of T lymphocytes was assessed according to the same procedure as in the A of Example 5 except that the concatameric fusion dimeric proteins, instead of the simple fusion dimeric proteins, were used, not separately but in combinations of [CTLA4-CTLA4/Fc]₂+[TNFR2-TNFR2/Fc]_{2,} [CTLA4-CTLA4/Fc]₂+[CD2-CD2/Fc]₂ and [CTLA4-CTLA4/Fc]₂+[LAG3-LAG3/Fc]₂ along with [CTLA4-CTLA4/Fc]₂ alone as a control (FIG. 6d).

As shown in FIG. 6d, the combinations of [CTLA4-CTLA4/Fc]₂+[TNFR2-TNFR2/Fc]₂, [CTLA4-CTLA4/Fc]₂+[CD2-CD2/Fc]₂ and [CTLA4-CTLA4/Fc]₂+[LAG3-LAG3/Fc]₂ as well as [CTLA4-CTLA4/Fc]₂ alone inhibted T lymphocyte proliferation. Also, the concatameric fusion dimeric proteins were found to be more effective in inhibiting the proliferation of T lymphocytes when used in combinations of two than when separately used. In particular, the combinaton of [CTLA4-CTLA4/Fc]₂+[LAG3-LAG3/Fc]₂ displayed the strongest inhibitory effect on the proliferation of T lymphocytes.

### EXAMPLE 6

### Evaluation of the Reducing Effects of the Simple Fusion Dimeric Proteins or Concatameric Fusion Dimeric Proteins on Collagen-Induced Arthritis when the Proteins are used Separately or in Combination

A. The reducing effects of the simple fusion dimeric proteins on collagen-induced arthritis when the proteins are used separately

A purified type II collagen, Arthrogen-CIA adjuvant (Chondrex, USA), was dissolved in 0.05 M acetic acid in a concentration of 2 mg/ml and injected into the tail vein of DBA/1 mice in an amount of 100 μg per mouse to induce collagen-induced arthritis (CIA). After three weeks, boosting was carried out with an incomplete Freund's adjuvant (Difco, USA).

Three to four weeks after DBA/1 mice were immunized with 100 μg of type II collagen, the mice developed arthritis. Three to five days after the onset of arthritis, the mice had red swollen feet, and inflammatory arthritis persisted over three to four weeks. Although inflammation was subsided, joints were permanently stiffened. Based on the visual scoring system for evaluating arthritis severity, lised in Table 3, below, arthritis severity was examined for the onset of erythema and swelling in joints two or three times per week (a mean value was calculated from severity scores of five mice per test group).

TABLE 3
Visual scoring system for evaluating arthritis severity

| Severity score | Gross pathology |
|---|---|
| 0 | No evidence of erythema and swelling |
| 1 | Erythema and mild swelling confined |
| | to the ankle or mid-foot joint (tarsals) |
| 2 | Erythema and mild swelling extending |
| | from the ankle to the mid-foot |
| 3 | Erythema and moderate swelling extending |
| | from the ankle to the metatarsal joints |
| 4 | Erythema and severe swelling encompassing |
| | the ankle, leg and digits |

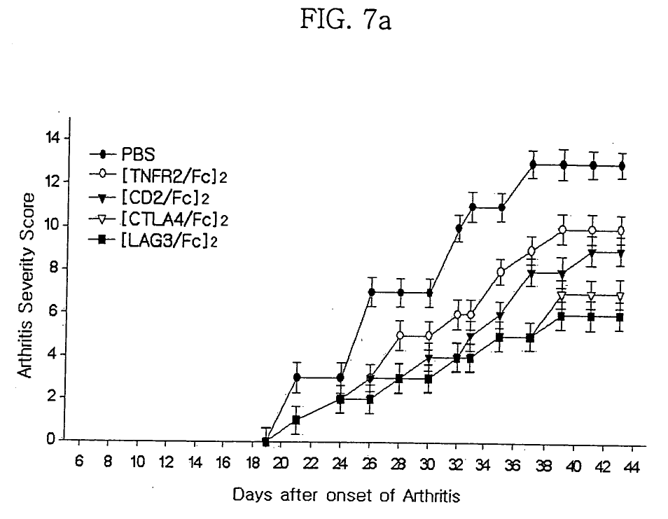

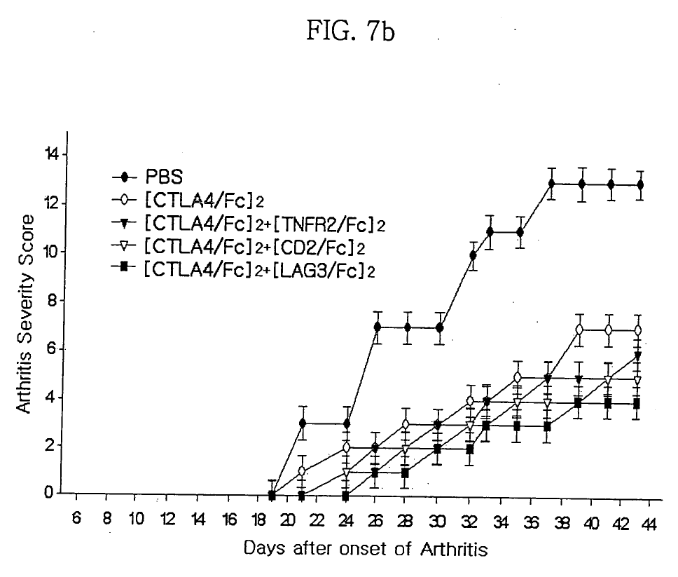

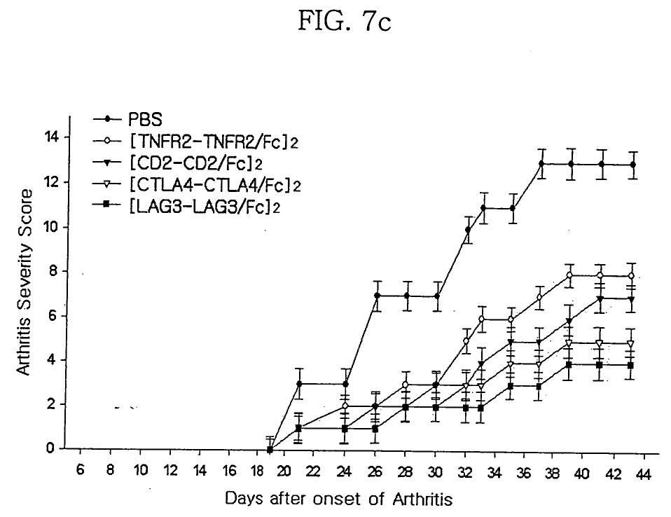

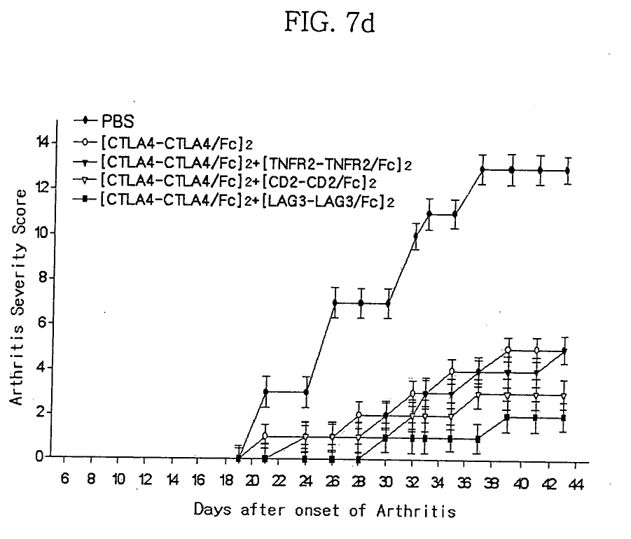

The simple fusion dimeric proteins, [TNFR2/Fc]₂, [CD2/Fc]₂, [CTLA4/Fc]₂ and [LAG3/Fc]₂, individually dissolved in PBS at a concentration of 200 μg/0.5 ml and injected intraperitoneally into the mice developing CIA. The dimeric forms of CD2/Fc, TNFR2/Fc, CTLA4/Fc and LAG3/Fc were injected in a dose of 10 μg into five mice from each test group every second day from day 19 to day 45, and the arthritis severity was evaluated (FIG. 7a).

As shown in FIG. 7a, when the simple fusion dimeric proteins were separately administered to the CIA-developing mice, they had a reduction of about 26-38% in arthritis severity based on severity measured on day 45 compared to a control group injected with PBS.

B. The reducing effects of the simple fusion dimeric proteins on CIA when the proteins are used in combination

The severity of arthritis in CIA mice was assessed according to the same procedure as in the A of Example 6 except that the simple fusion dimeric proteins were used not separately but in combinations of [CTLA4/Fc]₂, [CTLA4/Fc]₂+[TNFR2/Fc]₂, [CTLA4/Fc]₂+[CD2/Fc]₂ and [CTLA4/Fc]₂+[LAG3/Fc]₂ along with [CTLA4/Fc]₂ alone as a control (FIG. 7b).

As shown in FIG. 7b, the combinations of [CTLA4/Fc]₂+[TNFR2/Fc]_{2,} [CTLA4/Fc]₂+[CD2/Fc]₂ and [CTLA4/Fc]₂+[LAG3/Fc]₂ as well as [CTLA4/Fc]₂ alone reduced the severity of arthritis n mice. Also, the simple fusion dimeric proteins were found to be more effective in reducing the severity of arthritis in mice when administered in combinations of two than when separately administered.

C. The reducing effects of the concatameric fusion dimeric proteins on CIA when the proteins are used separately

The severity of arthritis in CIA mice was assessed according to the same procedure as in the A of Example 6 except that, instead of the simple fusion dimeric proteins, the concatameric fusion dimeric proteins, [TNFR2-TNFR2/Fc]₂, [CD2-CD2/Fc]₂, [CTLA4-CTLA4/Fc]₂ and [LAG3-LAG3/Fc]₂, were used separately (FIG. 7c).

As shown in FIG. 7c, the concatameric fusion dimeric proteins [TNFR2-TNFR2/Fc]₂, [CD2-CD2/Fc]_{2,} [CTLA4-CTLA4/Fc]₂ and [LAG3-LAG3/Fc]₂ all reduced the severity of arthritis in CIA mice. The concatameric fusion dimeric proteins used separately were found to be more effective in reducing the severity of arthritis in mice than the simple fusion dimeric proteins used separately, and displayed an arthritis-reducing effect similar to the combinations of the simple fusion dimeric proteins.

D. The reducing effects of the concatameric fusion dimeric proteins on CIA when the proteins are used in combination

The severity of arthritis in CIA mice was assessed according to the same procedure as in the A of Example 6 except that the concatameric fusion dimeric proteins, instead of the simple fusion dimeric proteins, were used, not separately but in combinations of [CTLA4-CTLA4/Fc]₂+[TNFR2-TNFR2/Fc]_{2,} [CTLA4-CTLA4/Fc]₂+[CD2-CD2/Fc]₂ and [CTLA4-CTLA4/Fc]₂+[LAG3-LAG3/Fc]₂ along with [CTLA4/Fc]₂ alone as a control (FIG. 7d).

As shown in FIG. 7d, the combinations of [CTLA4-CTLA4/Fc]₂+[TNFR2-TNFR2/Fc]₂, [CTLA4-CTLA4/Fc]₂+[CD2-CD2/Fc]₂ and [CTLA4-CTLA4/Fc]₂+[LAG3-LAG3/Fc]₂ as well as [CTLA4/Fc]₂ alone reduced the severity of arthritis in CIA mice. Also, the concatameric fusion dimeric proteins were found to be more effective in reducing the severity of arthritis in mice when used in combinations of two than when separately used.

### EXAMPLE 7

### Evaluation of the Therapeutic Effects of the Simple Fusion Dimeric Proteins or Concatameric Fusion Dimeric Proteins on Graft-Versus-Host Disease (GVHD) when the Proteins are used Separately or in Combination

A. The therapeutic effects of the simple fusion dimeric proteins on GVHD

8 to 12 week-old female C57BL/6 and BDF1 [(C57BL/6×DBA/2)F₁] mice, weighing 20 to 25 g, were used in this test, and were grown in a sterile filter-top microisolator. Recipient mice received bactrim one day before being transplanted with splenocytes from donor mice. BDF1 (H-2 Kb/d) recipient mice, which were irradiated with 700 cGy gamma-rays, were obtained from the microbiology lab of Yonsei University in Korea. Splenocytes from C57BL/6 donor mice were prepared using a medium containing 10% RPMI and 1% penicillin/streptomycin, and the cells were harvested by centrifugation at 400 g for 10 min.

In order to induce graft-versus-host disease (GVHD), 25×10⁶ viable splenocytes from allogeneic C57BL/6 donor mice (H-2 Kb) were transplanted into the gamma-ray-irradiated BDF1 recipient mice by a reverse injection method.

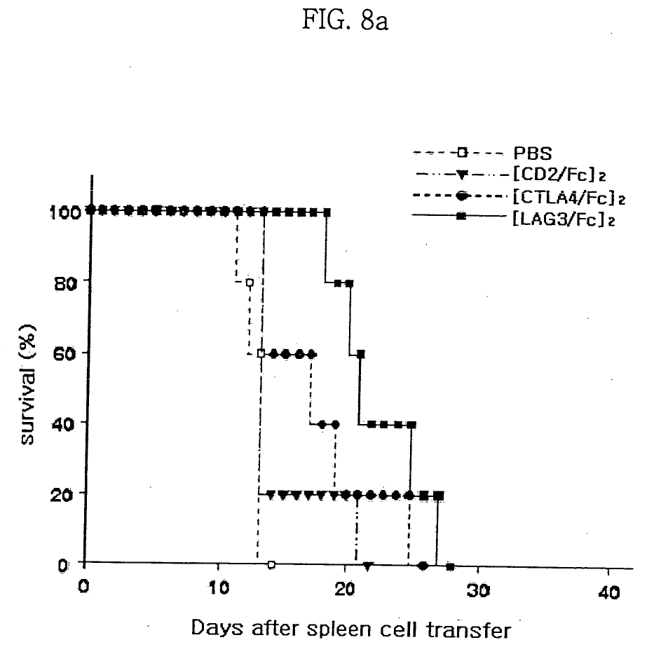

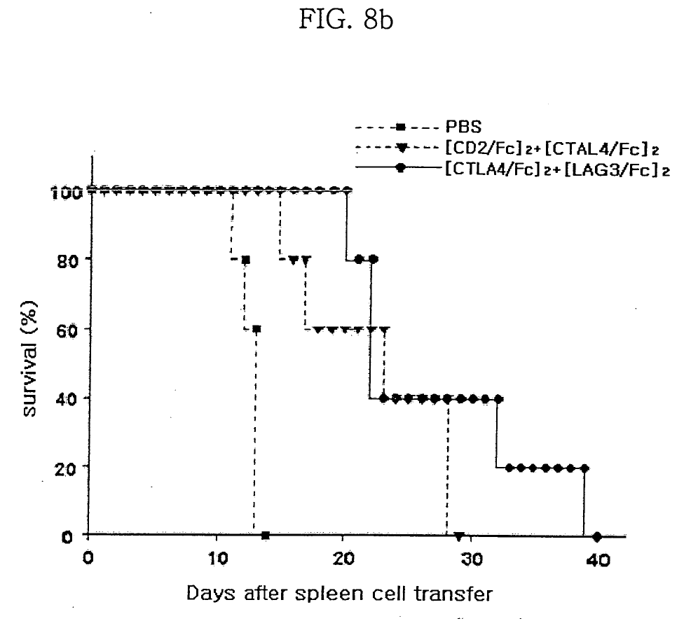

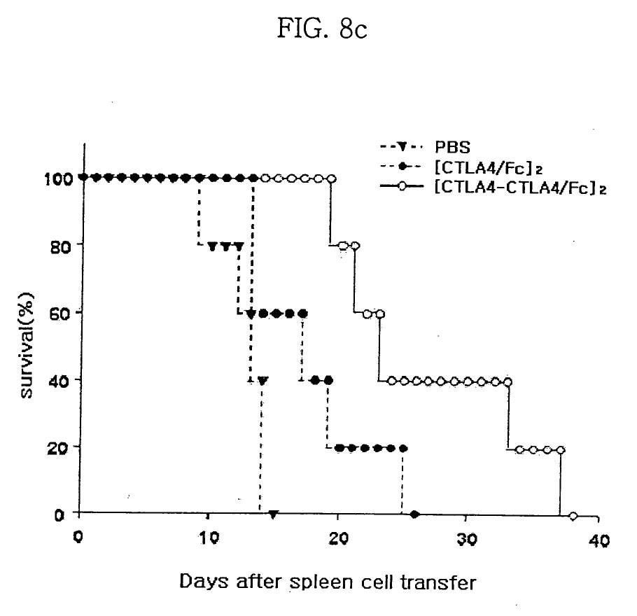

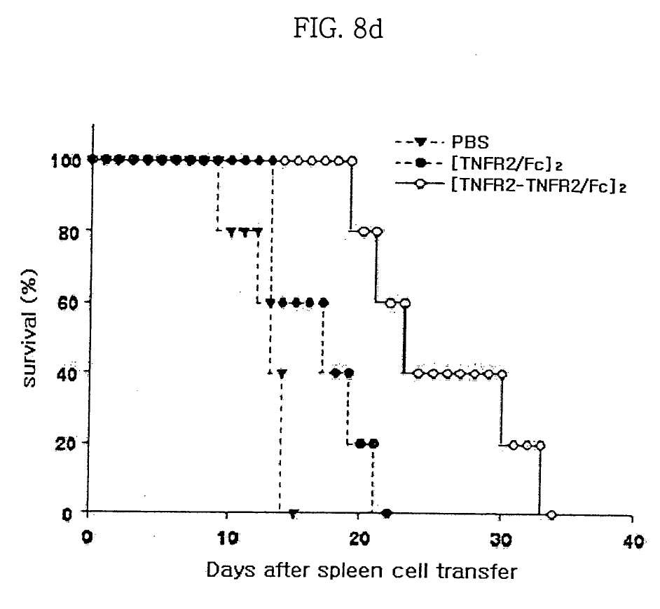

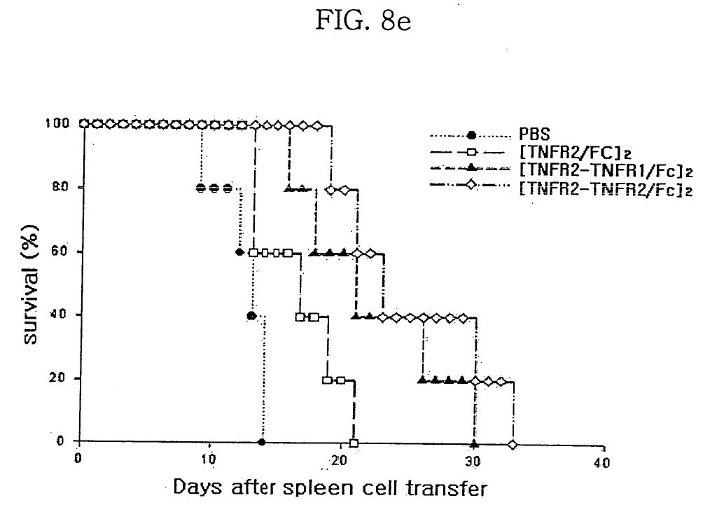

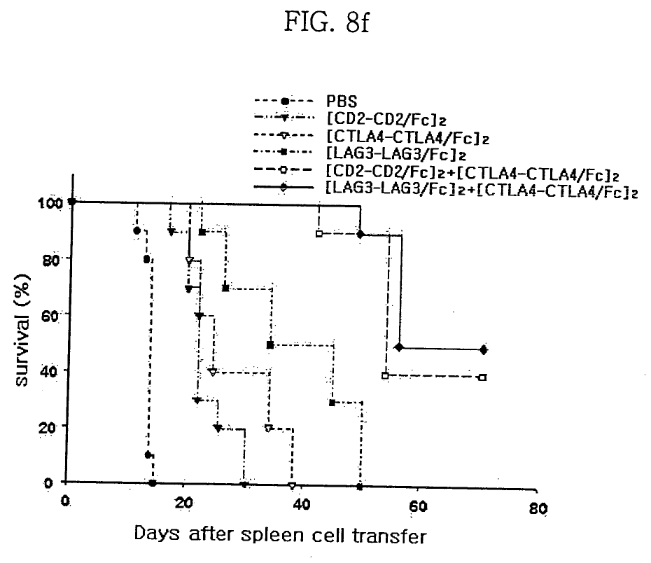

Then, the simple fusion dimeric proteins, [CD2/Fc]₂, [LAG3/Fc]₂ and [CTLA4/Fc]₂, were individually dissolved in PBS at a concentration of 200 μg/0.5 ml, and injected intraperitoneally into the recipient mice developing GVHD 0, 2, 4 and 6 days post-transplantation. Control recipient mice were administered with PBS. The recipient mice were monitored for survival by weighing the mice every two days (FIG. 8a).

As shown in FIG. 8a, control recipient mice rapidly lost weight due to developed GVHD, and displayed a reduction in the number of splenocytes due to proliferation of activated T lymphocytes from donor mice. About two weeks afer the transplantation of splenocytes into recipient mice, all control mice used in this test displayed severe weight loss, and eventually died. In contrast, when mice were administered with each of the simple fusion dimeric proteins, [CD2/Fc]₂, [LAG3/Fc]₂ and [CTLA4/Fc]_{2,} GVHD mortality was reduced in all mice compared to the control group. When the simple fusion dimeric proteins are separately administered to GVHD mice, [LAG3/Fc]₂ displayed the longest survival period of about four weeks and thus had the strongest immunosuppressive effect, followed by [CTLA4/Fc]₂ and then [CD2Fc]_{2,} whose separate administration also resulted in the improved survival of GVHD mice.

B. The therapeutic effects of the simple fusion dimeric proteins on GVHD when the proteins are used separately or in combination

The simple fusion dimeric proteins, [CD2/Fc]₂, [LAG3/Fc]₂ and [CTLA4/Fc]₂, were individually dissolved in PBS at a concentration of 200 μg/0.5 ml, and injected intraperitoneally into GVHD recipient mice 0, 2, 4 and 6 days post-transplantation. Likewise, combinations of the simple fusion dimeric proteins, [CD2Fc]₂+[CTLA4/Fc]₂ and [LAG3/Fc]₂+[CTLA4/Fc]₂, were individually dissolved in PBS at a concentration of 200 μg/0.5 ml, and injected intraperitoneally into GVHD recipient mice 0, 2, 4 and 6 days post-transplantation (FIG. 8b).

As shown in FIG. 8b, the combined administration of the simple fusion dimeric proteins resulted in higher viability of GVHD mice, compared to the results of the A of Example 7 in which the simple fusion dimeric proteins were administered separately. In particular, when GVHD mice were administered with the [LAG3/Fc]₂+[CTLA4/Fc]₂ combination, all individuals survived for over about 40 days, and this combination was found to most greatly reduce GVHD mortality. These results were obtained by measuring survival periods of ten mice from each group and computing mean values from the measured survival periods (Fable 4). These results indicate that the simple fusion dimeric proteins are more effective in treating GVHD when administered in combinations of two or more when administered separately.

TABLE 4
Comparison of the therapeutic effects of the simple fusion dimeric proteins
on GVHD when the proteins are used separately or in combination

| | | | | Survival | Mean survival |
|---|---|---|---|---|---|
| Immunosuppressive | Donor | Recipient | Mouse | period | period |
| agent (mg/kg/day) | mice | mice | numbers | (day) | (Mean ± SEM) |
| PBS | C57BL/6 | BDF1 | 10 | 11˜15 | 13.7 ± 1.06 |
| [CD2/Fc]₂ | C57BL/6 | BDF1 | 10 | 14˜22 | 15.7 ± 3.37 |
| [LAG3/Fc]₂ | C57BL/6 | BDF1 | 10 | 13˜26 | 18 ± 5.12 |
| [CTLA4/Fc]₂ | G57BL/6 | BDF1 | 10 | 19˜28 | 23.2 ± 3.49 |
| [CD2/Fc]₂ + | C57BL/6 | BDF1 | 10 | 16˜29 | 23.2 ± 5.71 |
| [CTLA4/Fc]₂ | | | | | |
| [LAG3/Fc]₂ + | C57BL/6 | BDF1 | 10 | 21˜40 | 28 ± 7.71 |
| [CTLA4/Fc]₂ | | | | | |

C. Comparison of the therapeutic effects of the simple fusion dimeric proteins and the concatameric fusion dimeric proteins on GVHD

(1) CTLA4

The simple fusion dimeric protein, [CTLA4/Fc]_{2,} was dissolved in PBS at a concentration of 200 μg/0.5 ml, and injected intraperitoneally into GVHD recipient mice 0, 2, 4 and 6 days post-transplantation. Likewise, the concatameric fusion dimeric protein, [CTLA4-CTLA4/Fc]₂, was dissolved in PBS at a concentration of 200 μg/0.5 ml, and injected intraperitoneally into GVHD recipient mice 0, 2, 4 and 6 days post-transplantation (FIG. 8c).

As shown in FIG. 8c, when GVHD recipient mice were administered with [CTLA4/Fc]₂ alone, the mice survived for a maximum of about 26 days. In contrast, when GVHD recipient mice were administered with [CTLA4-CTLA4/Fc]₂ alone, the mice survived for a maximum of about 38 days. These results were obtained by measuring survival periods of ten mice from each group and computing mean values from the measured survival periods (Table 5). These results indicate that concatameric fusion dimeric proteins are more effective in treating GVHD than are simple fusion dimeric proteins.

TABLE 5
Comparison of the therapeutic effects of the simple fusion dimeric
proteins and the concatameric fusion dimeric proteins on GVHD

| Immuno- | | | | | |
|---|---|---|---|---|---|
| suppressive | | Recip- | | Survival | Mean survival |
| agent | Donor | ient | Mouse | period | period |
| (mg/kg/day) | mice | mice | number | (day) | (Mean ± SEM) |
| PBS | C57BL/6 | BDF1 | 10 | 11˜15 | 13.7 ± 1.06 |
| [CTLA4/Fc]₂ | C57BL/6 | BDF1 | 10 | 14˜26 | 18.4 ± 4.70 |
| [CTLA4- | C57BL/6 | BDF1 | 10 | 19˜38 | 28.2 ± 8.12 |
| CTLA4/Fc]₂ | | | | | |

(2) TNFR2

The simple fusion dimeric protein, [TNFR2/Fc]_{2,} was dissolved in PBS at a concentration of 200 μg/0.5 ml, and injected intraperitoneally into GVHD recipient mice 0, 2, 4 and 6 days post-transplantation. Likewise, the concatameric fusion dimeric protein [TNFR2-TNFR2/Fc]_{2,} was dissolved in PBS at a concentration of 200 μg/0.5 ml, and injected intraperitoneally into GVHD recipient mice 0, 2, 4 and 6 days post-transplantation (FIG. 8d).

As shown in FIG. 8d, when GVHD recipient mice were administered with [TNFR2/Fc]₂ alone, the mice survived for a maximum of about 20 days. In contrast, when GVHD recipient mice were admit with [TNFR2-TNFR2/Fc]₂ alone, the mice survived for a maximum of about 35 days. These results indicate that concatameric fusion dimeric proteins are more effective in treating GVHD than simple fusion dimeric proteins.

D. Comparison of the therapeutic effects of [TNFR2/Fc]_{2,} [TNFR2/Fc]₂ and [TNFR2-TNFR1Fc]₂ on GVHD

The simple fusion dimeric protein, [TNFR2/Fc]₂, was dissolved in PBS at a concentration of 200 μg/0.5 ml, and injected intraperitoneally into GVHD recipient mice 0, 2, 4 and 6 days post-transplantation. Likewise, the concatameric fusion dimeric proteins, [TNFR2-TNFR2/Fc]₂ and [TNFR2-TNFR1/Fc]₂, were individually dissolved in PBS at a concentration of 200 μg/0.5 ml, and injected intraperitoneally into GVHD recipient mice 0, 2, 4 and 6 days post-transplantation (FIG. 8e).

As shown in FIG. 8e, when GVHD recipient mice were administered with [TNFR2/Fc]₂ alone, the mice survived for a maximum of about 20 days. In contrast, when GVHD recipient mice were administered with [TNFR2-TNFR1/Fc]₂ alone and [TNFR2-TNFR2/Fc]₂ alone, the mice survived for a maximum of about 30 days and a maximum of about 35 days, respectively. These results indicate that concatameric fusion dimeric proteins are more effective in treating GVHD than are simple fusion dimeric proteins. Also, compared to [TNFR2-TNFR1/Fc]₂, [TNFR2-TNFR2/Fc]₂ showed almost similar effects but was found to have stronger immunosuppressive effects.

E. The therapeutic effects of the concatameric fusion dimeric proteins on GVHD when the proteins are administered separately or in combination

The concatameric fusion dimeric proteins, [CD2-CD2Fc]₂, [LAG3-LAG3/Fc]₂, [CTLA4-CTLA4/Fc]₂ and [TNFR2-TNFR1/Fc]₂, were individually dissolved in PBS at a concentration of 200 μg/0.5 ml, and injected intraperitoneally into GVHD recipient mice 0, 2, 4 and 6 days post-transplantation. Likewise, combinations of the concatameric fusion dimeric proteins, [CD2-CD2/Fc]₂+[CTLA4-CTLA4/Fc]₂ and [LAG3-LAG3/Fc]₂+[CTLA4-CTLA4/Fc]₂, were individually dissolved in PBS at a concentration of 200 μg/0.5 ml, and injected intraperitoneally into GVHD recipient mice 0, 2, 4 and 6 days post-transplantation (FIG. 8f).

As shown in FIG. 8f, control mice displayed 100% mortality after about two weeks (Table 6), and these results are similar to the above results. Similar to the results of the B of Example 7 in which simple fusion dimeric proteins are administered, the concatameric fusion dimeric proteins were found to be more effective in improving the survival of GVHD mice when administered in combination than when administered separately. The combined administration of concatameric fusion dimeric proteins, [CD2-CD2/Fc]₂+[CTLA4-CTLA4/Fc]₂ and [LAG3-LAG3/Fc]₂+[CTLA4-CTLA4/Fc], resulted in survival rates of 40% and 50%, respectively, even about ten weeks afer the injection of splenocytes. These results indicate that the concatameric fusion dimeric proteins are more effective in treating GVHD when administered in combinations of two or more than when administered separately.

TABLE 6
Comparison of the therapeutic effects of the concatameric fusion dimeric proteins
on GVHD when the proteins are administered separately or in combination

| | | | | Survival | Mean survival |
|---|---|---|---|---|---|
| Immunosuppressive | Donor | Recipient | Mouse | period | period |
| agent (mg/kg/day) | mice | mice | number | (day) | (Mean ± SEM) |
| PBS | C57BL/6 | BDF1 | 10 | 11˜15 | 13.7 ± 4.3 |
| [CD2-CD2/Fc]₂ | C57BL/6 | BDF1 | 10 | 19˜28 | 21.4 ± 5.6 |
| [TNFR2-TNFR2/Fc]₂ | C57BL/6 | BDF1 | 10 | 20˜34 | 26.2 ± 6.1 |
| [TNFR2-TNFR1/Fc]₂ | C57BL/6 | BDF1 | 10 | 18˜31 | 23.6 ± 5.4 |
| [CTLA4-CTLA4/Fc]₂ | C57BL/6 | BDF1 | 10 | 19˜38 | 28.2 ± 8.2 |
| [LAG3-LAG3/Fc]₂ | C57BL/6 | BDF1 | 10 | 22˜50 | 34.6 ± 10.6 |
| [CD2-CD2/Fc]₂ + | C57BL/6 | BDF1 | 10 | >44 | >100 |
| [CTLA4-CTLA4/Fc]₂ | | | | | |
| [LAG3-LAG3/Fc]₂ + | C57BL/6 | BDF1 | 10 | >50 | >100 |
| [CTLA4-CTLA4/Fc]₂ | | | | | |

The Ig fusion proteins according to the present invention were all found to inhibit the activation of T lymphocytes. In particular, the concatameric fusion dimeric proteins had stronger inhibitory effects than the simple fusion dimeric proteins. In addition, both the simple fusion and concatameric fusion dimeric proteins were found to be more effective in suppressing the activation of T lymphocytes when administered in combination than when administered separately.

1. A pharmaceutical composition for treating immunological disorders by inhibiting activation of T lymphocytes, comprising, as active ingredients, two or more substances selected from the group consisting of: a substance capable of blocking binding of an MHC Class II molecule and a receptor thereof, a substance capable of blocking binding of a costimulatory molecule and a receptor thereof, a substance capable of blocking binding of an adhesion molecule and a receptor thereof, and a substance capable of blocking binding of a cytokine and a receptor thereof.

2. The pharmaceutical composition for treating immunological disorders according to claim 1, wherein the substance capable of blocking the binding of the MHC Class II molecule and CD4 is selected from the group consisting of (1) an antibody to the MHC Class II molecule; (2) a simple fusion monomeric protein formed by linkage of a soluble extracellular domain of LAG3 to a hinge region of an Fc fragment of an immunoglobulin molecule; (3) a simple fusion dimeric protein in which two molecules of the simple fusion monomeric protein are joined by intermolecular disulfide bonds in the hinge region; (4) a concatameric fusion monomeric protein formed by linkage of an N-terminus of a soluble extracellular domain of the LAG3, linked to the hinge region of the simple fusion monomeric protein, to a C-terminus of a soluble extracellular domain of another LAG3 molecule; (5) a concatameric fusion dimeric protein in which two molecules of the concatameric fusion monomeric protein are joined by intermolecular disulfide bonds in the hinge region; and (6) glycosylated forms of the proteins according to (2) to (5).

3. The pharmaceutical composition for treating immunological disorders according to claim 1, wherein the costimulatory molecule is selected from the group consisting of B7, CD154, CD70, 0X40L, ICOS-L, 4-1BBL, HVEM, FASL and PDL, and the receptor thereof is selected from the group consisting of CD28 and CTLA4, CD40, CD27, 0X40, ICOS, 4-1BB, LIGHT, FAS and PD-1.

4. The pharmaceutical composition for treating immunological disorders according to claim 3, wherein the substance capable of blocking the binding of the B7 molecule and the CD28 is selected from the group consisting of (1) an antibody to the B7 molecule; (2) a simple fusion monomeric protein formed by linkage of a soluble extracellular domain of the CTLA4 to a hinge region of an Fc fragment of an immunoglobulin molecule; (3) a simple fusion dimeric protein in which two molecules of the simple fusion monomeric protein are joined by intermolecular disulfide bonds in the hinge region; (4) a concatameric fusion monomeric protein formed by linkage of an N -terminus of a soluble extracellular domain of the CTLA4, linked to the hinge region of the simple fusion monomeric protein, to a C-terminus of a soluble extracellular domain of another CTLA4 molecule; (5) a concatameric fusion dimeric protein in which two molecules of the concatameric fusion monomeric protein are joined by intermolecular disulfide bonds in the hinge region; and (6) glycosylated forms of the proteins according to (2) to (5).

5. The pharmaceutical composition for treating the immunological disorder according to claim 1, wherein the adhesion molecule is LFA-3, ICAM-1 or VCAM-1, and the receptor thereof is CD2, LFA-1 or VLA-4.

6. The pharmaceutical composition for treating immunological disorders according to claim 5, wherein the substance capable of blocking the binding of the LFA-3 and the CD2 is selected from the group consisting of (1) an antibody to the LFA-3; (2) a simple fusion monomeric protein formed by linkage of a soluble extracellular domain of the CD2 to a hinge region of an Fc fragment of an immunoglobulin molecule; (3) a simple fusion dimeric protein in which two molecules of the simple fusion monomeric protein are joined by intermolecular disulfide bonds in the hinge region; (4) a concatameric fusion monomeric protein formed by linkage of an N-terminus of a soluble extracellar domain of the CD2, linked to the hinge region of the simple fusion monomeric protein, to a C-terminus of a soluble extracellular domain of another CD2 molecule; (5) a concatameric fusion dimeric protein in which two molecules of the concatameric fusion monomeric protein are joined by intermolecular disulfide bonds in the hinge region; and (6) glycosylated forms of the proteins according to (2) to (5).

7. The pharmaceutical composition for treating immunological disorders according to claim 1, wherein the cytokine is selected from the group consisting of IL-1, IL-2, IL-3, IL-4, IL-5, IL-6, IL-7, TNF, TGF, IFN, GM-CSF, G-CSF, EPO, TPO or M-CSF, and the receptor thereof is IL-1R, IL-2R, IL-3R, IL-4R, IL-5R, IL-6R, IL-7R, TNFR, TGFR, IFNR, INF-α R, INF-β R, INF-γ R, GM-CSFR, G-CSFR EPOR, cMp1 and gp13O.

8. The pharmaceutical composition for treating immunological disorders according to claim 7, wherein the substance capable of blocking the binding of the TNF and the TNFR is selected from the group consisting of (1) an antibody to the TNF; (2) a simple fusion monomeric protein formed by linkage of a soluble extracellular domain of the TNFR to a hinge region of an Fc fragment of an immunoglobulin molecule; (3) a simple fusion dimeric protein in which two molecules of the simple fusion monomeric protein are joined by intermolecular disulfide bonds in the hinge region; (4) a concatameric fusion monomeric protein formed by linkage of an N-terminus of a soluble extracellar domain of the TNFR, linked to the hinge region of the simple fusion monomeric protein, to a C-terminus of a soluble extracellular domain of another TNFR molecule; (5) a concatameric fusion dimeric protein in which two molecules of the concatameric fusion monomeric protein are joined by intermolecular disulfide bonds in the hinge region; and (6) glycosylated forms of the proteins according to (2) to (5).

9. The pharmaceutical composition for treating immunological disorders according to claim 1, wherein the immunological disorder is an autoimmune disease or a transplantation rejection.

10. The pharmaceutical composition for treating immunological disorders according to claim 9, wherein the autoimmune disease is selected from the group consisting of rheumatoid arthritis, multiple sclerosis, myasthenia gravis, Grave's disease, Hashimoto's thyroiditis, Addison's disease, vitilligo, scleroderma, Goodpasture syndrome, Becet's disease, Crohn's disease, ankylosing spondylitis, uveitis, thrombocytopenic purpura, pemphigus vulgaris, childhood diabetes, autoimmune anemia, cryoglobulinemia, adrenoleukodystrophy (AID), and systemic lupus erythematosus (SLE).
